(19) United States
(12) Patent Application Publication
(10) Pub. No.: US 2026/0144520 A1
(43) Pub. Date: May 28, 2026

(54) MULTI-ARRAY SCANNER

(71) Applicant: FUJIFILM SONOSITE, INC., Bothell, WA (US)

(72) Inventors: Jimin Zhang, Bellevue, WA (US); Craig Chamberlain, Seattle, WA (US); Katsuya Yamamoto, Kaisei (JP); Thomas Endres, Sagle, ID (US); Richard Kelly, Bothell, WA (US)

(21) Appl. No.: 19/422,191

(22) Filed: Dec. 16, 2025

Related U.S. Application Data
(63) Continuation-in-part of application No. 18/957,403, filed on Nov. 22, 2024.

(51) Int. Cl. A61B 8/00; B06B 1/02; B06B 1/06; G01S 15/89

(52) U.S. Cl. CPC A61B 8/4488; A61B 8/54; B06B 1/0292; B06B 1/0622; G01S 15/8927; B06B 2201/76

(57) ABSTRACT

An ultrasound device, ultrasound system and method for performing the same with a multi-array scanner are disclosed. In some embodiments, an ultrasound device includes an array that has a plurality of rows of ultrasonic transducer elements, with the plurality of rows of transducer elements having a first row of transducer element sub-arrays and two or more outer rows of transducer element sub-arrays. The two or more outer rows have at least one row on two opposite sides of the first row of transducer element sub-arrays, and transducer element sub-arrays in first and second rows of transducer element sub-arrays of the one or more outer rows have heights and widths that are different from each other, with the height of each transducer element sub-array corresponding to a lateral dimension and the width corresponding to an elevation dimension perpendicular to the lateral dimension.

### RELATED APPLICATION

The present application is a continuation-in-part of and claims the benefit of U.S. patent application Ser. No. 18/957,403, filed on Nov. 22, 2024, and entitled “DETERMINING PORT HEALTH WITH ULTRASOUND”, which is incorporated herein by reference in its entirety.

### FIELD

Embodiments disclosed herein relate to ultrasound systems. More specifically, embodiments disclosed herein are related to a multi-array ultrasound transducer having one or more sub-arrays of one or more piezoelectric transducers (PZTs), piezoelectric micromachined ultrasonic transducers (PMUTs), and capacitive micromachined ultrasonic transducers (CMUTs).

### BACKGROUND

Many medical conditions require the repeated insertion and/or removal of fluid into a patient's body, such as the use of chemotherapy for cancer treatment, infection that requires long-term intravenous (IV) antibiotics, kidney failure that requires dialysis, inflammatory bowel disease (IBD) that requires parenteral IV nutrition, diseases that require multiple blood transfusions (e.g., liver disease, sickle-cell anemia, etc.), and the like. To reduce the impact on the patient anatomy that can be caused by the repeated use of a needle to insert and/or remove the fluid, the patient may be fitted with a port.

A port is an implantable reservoir with a tube attached to it that can be inserted into a blood vessel. The reservoir portion of the port is placed just beneath the patient's skin, and the tube can be inserted into the patient's blood vessel (e.g., vein). Fluid can then be inserted and/or removed by inserting a needle into the port, rather than directly into the blood vessel, thus eliminating painful needle sticks into the blood vessel, and the damage caused by the needle sticks. However, ports have high failure rates, typically up to 50%. Failed ports can prevent or delay a procedure, become a source of infection, and cause additional cost and patient harm. For instance, a failed port may need to be replaced, which can require the patient to undergo an additional surgery with anesthesia.

### SUMMARY

An ultrasound device, ultrasound system and method for performing the same with a multi-array scanner are disclosed. In some embodiments, an ultrasound device includes: a lens; and array coupled to the lens; and a controller. The array has a plurality of rows of ultrasonic transducer elements, with the plurality of rows of transducer elements having a first row of transducer element sub-arrays and two or more outer rows of transducer element sub-arrays. The two or more outer rows have at least one row on two opposite sides of the first row of transducer element sub-arrays, and transducer element sub-arrays in first and second rows of transducer element sub-arrays of the one or more outer rows have heights and widths that are different from each other, with the height of each transducer element sub-array corresponding to a lateral dimension and the width corresponding to an elevation dimension perpendicular to the lateral dimension. The controller is coupled to the array and configured to control the first row of transducer element sub-arrays and two or more outer rows of transducer element sub-arrays to operate at a same time or at different times.

In some other embodiments, the ultrasound device includes a lens and a multi-array transducer coupled to the lens and having a plurality of transducer sub-arrays, where at least first and transducer arrays of the plurality of transducer arrays have heights and widths that are different from each other. The height of each transducer array corresponds to a lateral dimension across the array and the width transducer array corresponding to an elevation dimension perpendicular to the lateral dimension. The ultrasound device also includes a controller coupled to the array and configured to control the plurality of transducer sub-arrays to operate at a same time or at different times and to perform harmonic imaging with selectable bandwidths and center frequencies of first and second transducer sub-arrays to cause a configurable overlap of the bandwidths, the first transducer sub-array controlled to operate at a first ultrasound frequency and to transmit ultrasound and the second transducer sub-array controlled to operate at a second ultrasound frequency, different from the first ultrasound frequency and to receive reflections of the ultrasound.

Other aspects and advantages of the embodiments will become apparent from the following detailed description taken in conjunction with the accompanying drawings which illustrate, by way of example, the principles of the described embodiments.

### BRIEF DESCRIPTION OF THE DRAWINGS

The appended drawings illustrate examples and are, therefore, exemplary embodiments and not considered to be limiting in scope.

FIG. 1 illustrates some embodiments of an ultrasound system in a environment for determining port health with ultrasound during an ultrasound examination.

FIG. 2 illustrates some embodiments of an ultrasound system from FIG. 1.

FIG. 3 illustrates some embodiments of a system in an environment for determining port health with ultrasound.

FIG. 4 illustrates some embodiments of a system for determining port health with ultrasound.

FIG. 5 illustrates some embodiments of a user interface of a system for determining port health with ultrasound.

FIG. 6 illustrates some embodiments of example configurations of a reconfigurable wearable ultrasound scanner for determining port health with ultrasound.

FIG. 7 illustrates an environment with an example ultrasound scanner used for in-plane needle insertion.

FIG. 8 illustrates an environment with an example ultrasound scanner used for out-of-plane needle insertion.

FIG. 9 illustrates some embodiments of an example multi-array transducer for determining port health with ultrasound.

FIG. 10 illustrates some embodiments of example characteristics of a multi-array transducer for determining port health with ultrasound.

FIG. 11 illustrates some embodiments of a multi-array transducer for determining port health with ultrasound.

FIG. 12 illustrates some embodiments of a multi-array transducer for determining port health with ultrasound.

FIG. 13 illustrates some embodiments of tuning impedances for transducer arrays for determining port health with ultrasound.

FIG. 14 illustrates some embodiments of array configurations for a multi-array transducer of an ultrasound scanner for determining port health with ultrasound.

FIG. 15 illustrates some embodiments of a machine-learning architecture used to train a machine-learned model.

FIG. 16 illustrates some embodiments of a machine-learned model using a CNN.

FIG. 17 illustrates some embodiments of an example computing device for determining port health with ultrasound.

FIG. 18 illustrates some embodiments of a method for determining port health with ultrasound.

FIG. 19 illustrates some other embodiments of a method for determining port health with ultrasound.

FIG. 20 illustrates yet some other embodiments of a method for determining port health with ultrasound.

FIG. 21 illustrates some embodiments of an example multi-array transducer.

FIG. 22 illustrates some embodiments of example characteristics of a multi-array transducer.

FIG. 23 illustrates some embodiments of example array configurations for a multi-array transducer.

FIG. 24 illustrates some embodiments of an example calibration system for a multi-array transducer.

FIG. 25 illustrates some embodiments of an example method for controlling an ultrasound system.

FIG. 26 illustrates some embodiments of an example method for controlling an ultrasound system.

FIG. 27 illustrates some embodiments of an example method for controlling an ultrasound system.

### DETAILED DESCRIPTION

In the following description, numerous details are set forth to provide a more thorough explanation of the present invention. It will be apparent, however, to one skilled in the art, that the present invention may be practiced without these specific details. In other instances, well-known structures and devices are shown in block diagram form, rather than in detail, in order to avoid obscuring the present invention.

Monitoring of a port can be done with ultrasound. Ultrasound systems can generate ultrasound images by transmitting sound waves at frequencies above the audible spectrum into a body, receiving echo signals caused by the sound waves reflecting from internal body parts, and converting the echo signals into electrical signals for image generation. Ultrasound systems for port monitoring rely on a clinician (e.g., sonographer, nurse, doctor, or other trained operator) to acquire the ultrasound data. This monitoring can require repeated visits to a care facility or imaging facility and be inconvenient and expensive for the patient.

Accordingly, embodiments described herein include systems, devices, and methods for determining port health with ultrasound. In some embodiments, an ultrasound system includes a wearable ultrasound scanner that includes a patch configured for placement on a patient's skin over a port. The ultrasound system can implement one or more machine-learned models to generate a health status report that includes a prediction of when the port will fail. The wearable ultrasound scanner can include a multi-array transducer. In some embodiments, the multi-array transducer includes arrays comprised of lead zirconate titanate (PZT) array elements, capacitive micromachined ultrasonic transducer (CMUT) array elements, and/or piezoelectric micromachined ultrasonic transducer (PMUT) array elements. These and other aspects of determining port health with ultrasound are described in more detail below.

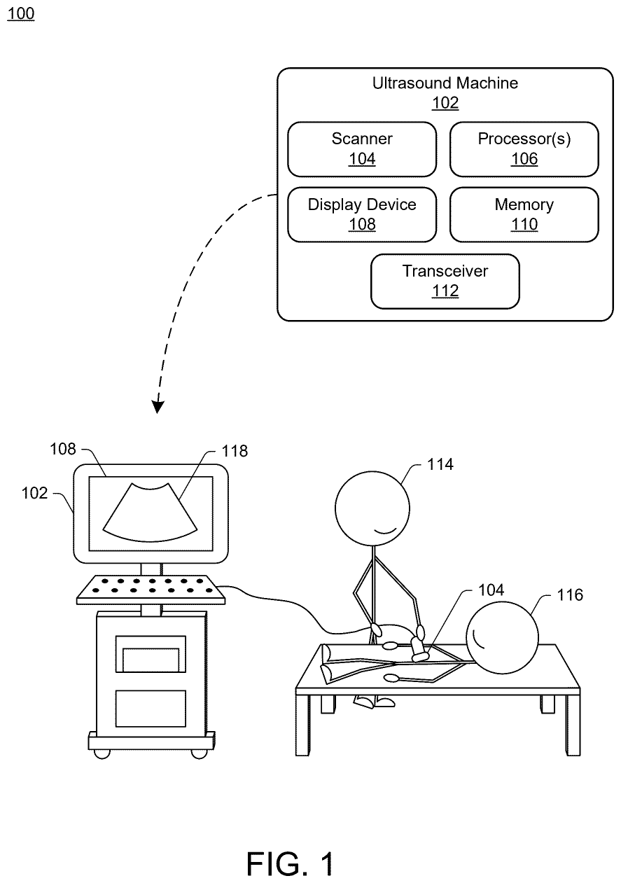

FIG. 1 illustrates an ultrasound system in an environment 100 for determining port health with ultrasound. Many medical conditions require the repeated insertion and/or removal of fluid into a patient's body, such as the use of chemotherapy for cancer treatment, infection that requires long-term intravenous (IV) antibiotics, kidney failure that requires dialysis, inflammatory bowel disease (IBD) that requires parenteral IV nutrition, diseases that require multiple blood transfusions (e.g., liver disease, sickle-cell anemia, etc.), and the like. To reduce the impact on the patient anatomy that can be caused by the repeated use of a needle to insert and/or remove the fluid, the patient may be fitted with a port.

The ultrasound system in FIG. 1 includes an ultrasound machine 102 and an ultrasound scanner 104. The ultrasound machine 102 generates high-frequency sound waves (e.g., ultrasound) and imaging data based on the ultrasound reflecting off a patient anatomy/body structure and/or an interventional instrument (e.g., a needle that is inserted into a port). The ultrasound machine 102 includes various components, some of which include the scanner 104, one or more processors 106, a display device 108, a memory 110, and a transceiver 112.

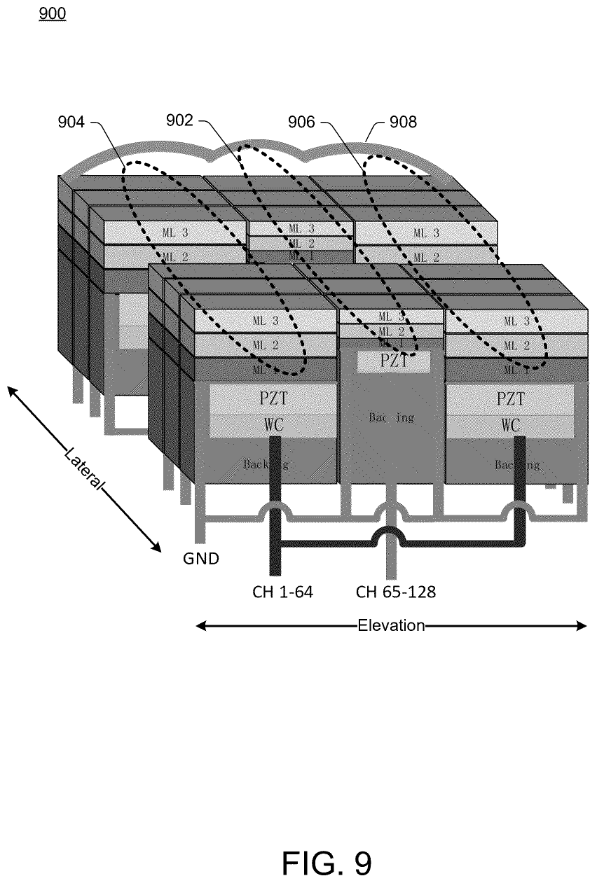

A user 114 (e.g., nurse, ultrasound technician, operator, sonographer, clinician, etc.) directs the scanner 104 toward a patient 116 to non-invasively scan internal bodily structures (e.g., patient anatomies such as organs, tissues, bones, etc.) of the patient 116, a port, an interventional instrument, etc., for testing, diagnostic, therapeutic, or procedural reasons, including determining port health. In some embodiments, the scanner 104 includes an ultrasound transducer array and electronics communicatively coupled to the ultrasound transducer array to transmit ultrasound signals to the patient's anatomy and receive ultrasound signals reflected from the patient's anatomy. In some embodiments, the scanner 104 is an ultrasound scanner, which can also be referred to as an ultrasound probe or transducer. In some embodiments, the scanner 104 is a multi-array scanner. For instance, a multi-array scanner in accordance with some embodiments can include one or more of the arrays described in U.S. patent application Ser. No. 18/613,694 filed on Mar. 22, 2024, entitled Multi-Dimensional and Multi-Frequency Ultrasound Transducers to Zhang et al., the disclosure of which is incorporated herein by reference in its entirety. A multi-array scanner in accordance with some embodiments can include one or more of the arrays described in U.S. patent application Ser. No. 17/561,313 filed on Dec. 23, 2021, entitled Array Architecture and Interconnection for Transducers to Li et al., the disclosure of which is incorporated herein by reference in its entirety. Further, multi-array scanners for determining port health with ultrasound are discussed below in more detail with respect to FIGS. 9-14.

The display device 108 is coupled to the processor 106, which can include any suitable processor, number of processors, or processor system, such as one or more central processing units (CPUs), graphics processing units (GPUs), vector processors, Reduced Instruction Set Computer (RISC) processors, Reduced Instruction Set Computer (CISC) processors, very long instruction word (VLIW) processors, etc. The processor 106 can execute instructions stored on memory 110 to perform operations disclosed herein for determining port health with ultrasound. For example, the processor 106 can process the reflected ultrasound signals to generate ultrasound data, including an ultrasound image. The display device 108 is configured to generate and display an ultrasound image (e.g., ultrasound image 118) of the anatomy and/or interventional instrument (e.g., a port or needle) based on the ultrasound data generated by the processor 106 from the reflected ultrasound signals detected by the scanner 104. In some embodiments, the ultrasound data includes the ultrasound image 118 or data representing the ultrasound image 118. The transceiver 112 can be configured to transmit, e.g., over a network maintained by a care facility, the ultrasound data and/or any data related to the ultrasound examination, such as medical worksheet data, a health status report of a port, etc., to a medical archiver (e.g., a vendor neutral archive (VNA)). In some embodiments, the transceiver 112 can receive data from the medical archiver, such as patient history data or previous examination data.

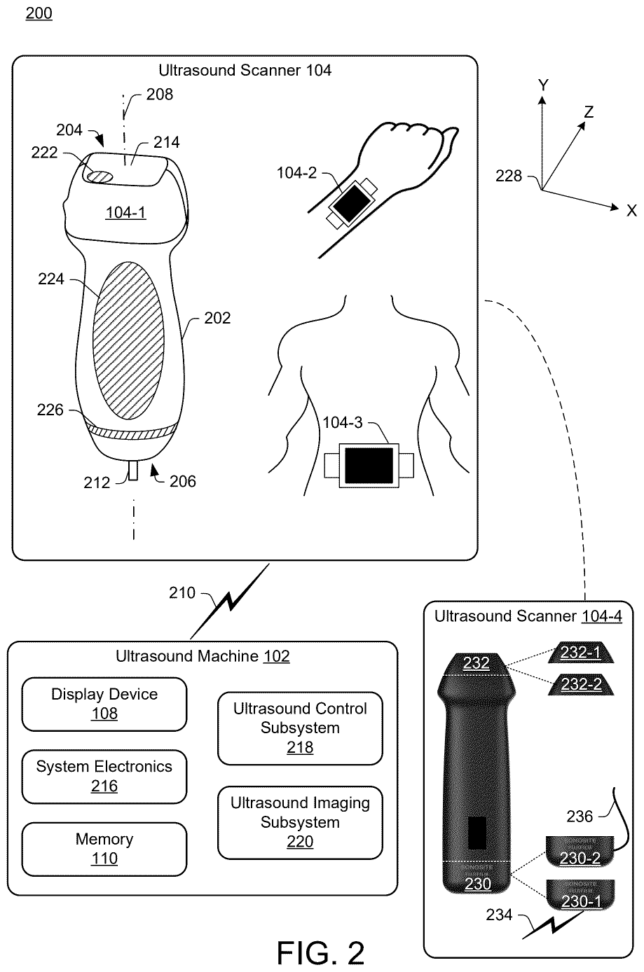

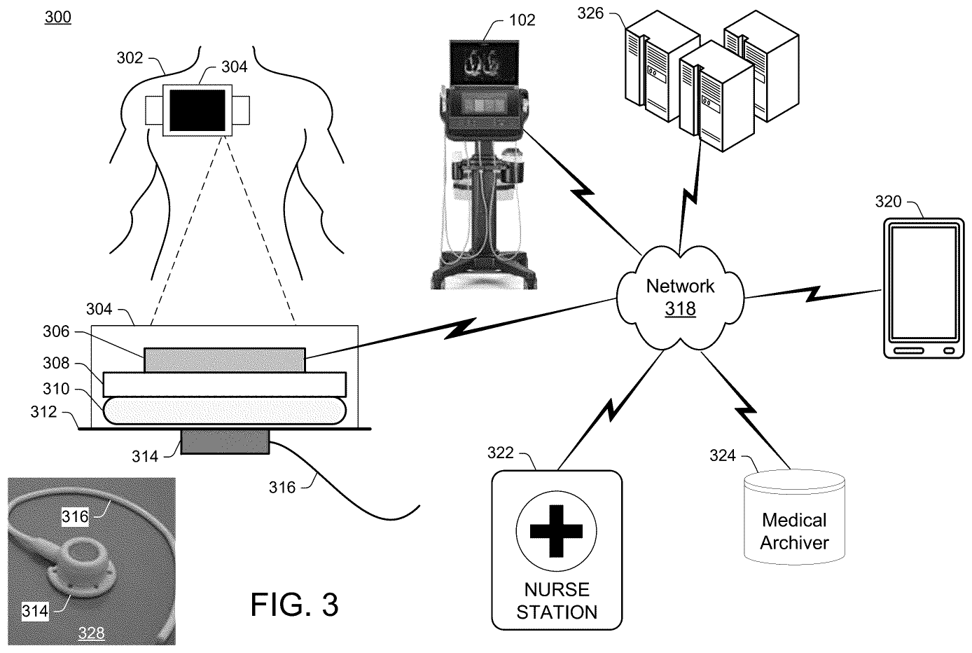

FIG. 2 illustrates an example implementation 200 of the ultrasound system illustrated in the environment 100 of FIG. 1. In the implementation 200, the scanner 104 (e.g., ultrasound scanner) can be any suitable type of ultrasound scanner. In some embodiments, the scanner 104 includes a scanner 104-1 configured for handheld operation, e.g., external to a patient's body. Other embodiments of the scanner 104, including scanner 104-2 and 104-3, include wearable ultrasound scanners (e.g., patch-based ultrasound scanners), that can be worn by a patient for testing, diagnostic, therapeutic, or procedural reasons, including long term monitoring for determining port health with ultrasound, and are discussed below in more detail with respect to FIGS. 3-6. Another example of the scanner 104 includes the ultrasound scanner 104-4, which is configured for handheld operation like the scanner 104-1 and includes removably attachable ultrasound arrays and removably attachable electronics for wired and/or wireless operation, as discussed below in more detail.

The scanner 104-1 includes an enclosure 202 extending between a distal end portion 204 and a proximal end portion 206. The enclosure 202 includes a central axis 208 (e.g., longitudinal axis) that intersects the distal end portion 204 and the proximal end portion 206. The central axis 208 corresponds to an axial direction of the scanner 104-1. The scanner 104-1 is electrically coupled to an ultrasound imaging system (e.g., the ultrasound machine 102) via a coupling 210. In some embodiments, the coupling 210 includes a cable that is attached to the proximal end portion 206 of the scanner 104-1 by a strain-relief element 212. In some embodiments, the coupling 210 includes a wireless coupling so that the scanner 104-1 is wirelessly coupled to the ultrasound imaging system and communicates with the ultrasound imaging system via one or more wireless transmitters, receivers, or transceivers over a wireless connection or network (e.g., Bluetooth™, Wi-Fi™, etc.).

A transducer assembly 214 having one or more transducer elements is electrically coupled to system electronics 216 in the ultrasound machine 102. In operation, the transducer assembly 214 transmits ultrasound energy from the one or more transducer elements toward a subject and receives ultrasound echoes from the subject. The ultrasound echoes are converted into electrical signals by the transducer element(s) and electrically transmitted to the system electronics 216 in the ultrasound machine 102 for processing and generation of one or more ultrasound images.

Capturing ultrasound data from a subject using a transducer assembly (e.g., the transducer assembly 214) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than 15 Megahertz (MHz)) and/or high-frequency ultrasound (e.g., greater than or equal to 15 MHz). A particular frequency range to use can readily be determined based on various factors, including, for example, depth of imaging, desired resolution, and so forth.

In some embodiments, the system electronics 216 include one or more processors (e.g., the processor(s) 106 from FIG. 1), integrated circuits, application-specific integrated circuits (ASICs), Field Programmable Gate Arrays (FPGAs), and power sources to support functioning of the ultrasound machine 102. In some embodiments, the ultrasound machine 102 also includes an ultrasound control subsystem 218 having one or more processors. At least one processor, FPGA, or ASIC can cause electrical signals to be transmitted to the transducer(s) of the scanner 104 to emit sound waves and also receives electrical pulses from the scanner 104 that were created from the returning echoes. One or more processors, FPGAs, or ASICs can process the raw data associated with the received electrical pulses and form an image that is sent to an ultrasound imaging subsystem 220, which causes the image (e.g., the image 118 in FIG. 1) to be displayed via the display device 108. Thus, the display device 108 displays ultrasound images from the ultrasound data processed by the processor(s) of the ultrasound control subsystem 218.

In some embodiments, the ultrasound machine 102 also includes one or more user input devices (e.g., a keyboard, a cursor control device, a microphone, a camera, touchscreen, etc.) that input data and enable taking measurements from the display device 108 of the ultrasound machine 102. The ultrasound machine 102 can also include a disk storage device (e.g., computer-readable storage media such as read-only memory (ROM), a Flash memory, a dynamic random-access memory (DRAM), a NOR memory, a static random-access memory (SRAM), a NAND memory, and so on) for storing the acquired ultrasound data. In some embodiments, the disk storage device includes the memory 110, which is local to the ultrasound machine 102. Alternatively, the memory 110 used for storing the acquisition data can be remote, such as on a remote server communicatively connected to the ultrasound machine 102. In addition, the ultrasound machine 102 can include a printer that prints the image from the displayed data. To avoid obscuring the techniques described herein, such user input devices, disk storage device, and printer are not shown in FIG. 2.

In some embodiments, the ultrasound scanner 104-1 in the implementation 200 also includes one or more pressure sensors 222 on the lens of the scanner 104-1, and one or more pressure sensors 224 on the enclosure 202 of the scanner 104-1. The pressure sensors 222 and 224 can include in, on, or under a sensor region any suitable type of sensors for determining a pressure. In one example, the pressure sensors 222 and 224 includes capacitive sensors that can measure a capacitance, or change in capacitance, caused by a user's touch or proximity of touch, as is common in touchscreen technologies. The pressure sensors 222 and 224 can generate sensor data indicative of a touch or pressure. The sensor data can include a binary indicator that indicates the presence and absence of a touch on the sensor. For instance, a “1” for sensor data can indicate that a pressure is sensed at the pressure sensor, and a “0” for the sensor data can indicate that a pressure is not sensed at the pressure sensor. Additionally or alternatively, the sensor data can include a multi-level indicator that indicates an amount of pressure on the sensor, such as an integer scale from zero to five. For instance, a “0” can indicate that no pressure is detected at the sensor, and a “1” can indicate a small amount of pressure is detected at the sensor. A “2” can indicate a larger amount of pressure is detected at the sensor than a “1”, and a “5” can indicate a maximum amount of pressure is detected at the sensor.

The pressure sensors 222 and 224 are illustrated in FIG. 2 as ellipses for clarity, and generally can be of any suitable shape and size and generate sensor data indicating pressure at any suitable number of points. For instance, in some embodiments, the pressure sensors 222 cover an exterior surface of the lens of the scanner 104-1 and can be used to determine when the scanner is placed against a patient. Additionally or alternatively, the pressure sensors 224 can substantially cover the enclosure 202 of the scanner 104-1 and can be used to determine when a clinician grabs the scanner 104-1 for use in an ultrasound examination (e.g., the clinician has a suitable grip on the scanner 104-1 to perform the ultrasound examination). The ultrasound system can use the sensor data from one or both of the pressure sensors 222 and 224 to generate a trigger signal that can be used for determining port health with ultrasound. For instance, when the sensor data from one or both of the pressure sensors 222 and 224 is above a threshold level, and/or the sensor data from the pressure sensors 224 indicate a grip pattern indicative of a human operating the scanner, the system can generate a trigger signal. The trigger signal can be used to cause the ultrasound system to enable one or more machine-learned models to generate a health status of a port, including a prediction of when the port will fail. For instance, the ultrasound system can, based on the trigger signal, determine from the ultrasound data one or more of an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of the fluid or the additional fluid. One or more machine-learned models can make these determinations. Another machine-learned model can then process this data and generate the health status of the port, including a prediction of when the port will fail.

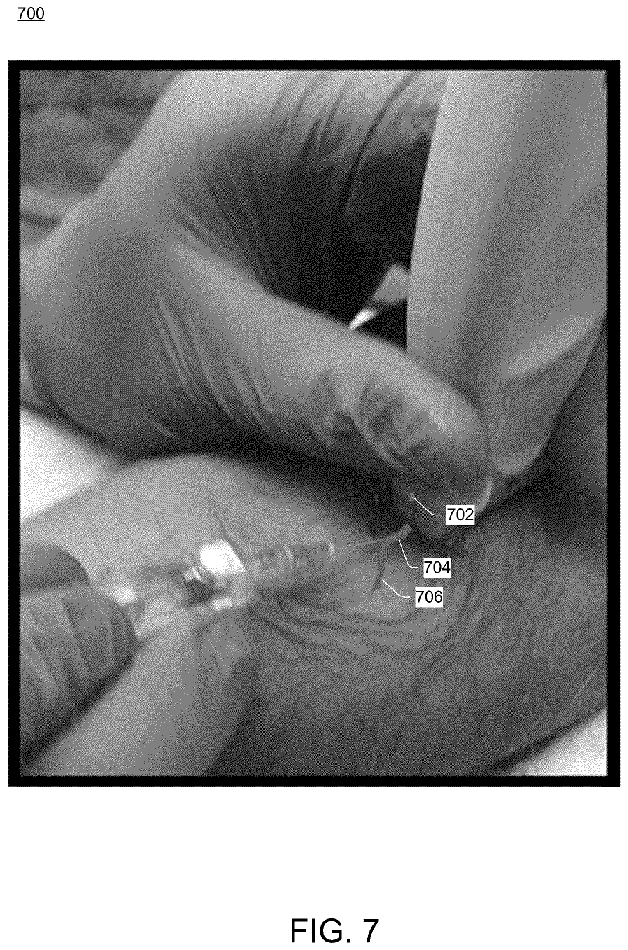

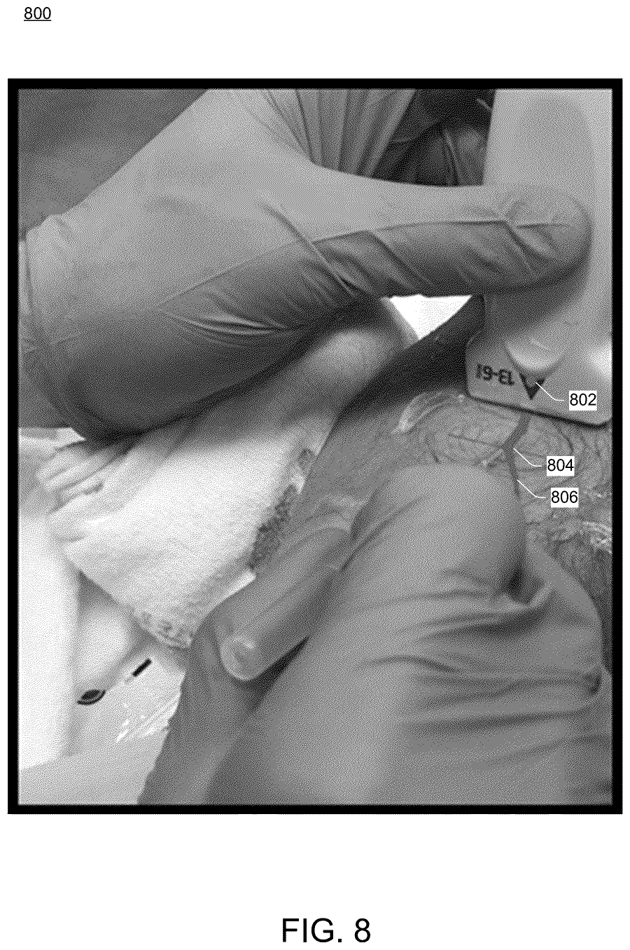

In some embodiments, the ultrasound system uses the trigger signal to enable one or more light sources (e.g., microelectromechanical systems (MEMS) lasers) for needle insertion guidance. For instance, the light sources can indicate a current position of the needle tip, under which array out of multiple arrays on the scanner the needle tip is positioned, indicate the position of a blood vessel, indicate an insertion point for the needle on the patient's skin, etc. The light sources can project light onto the patient's skin from the scanner, discussed below in more detail with respect to FIGS. 7 and 8.

In some embodiments, the scanner 104-1 includes an inertial measurement unit (IMU) 226 for generating positional data that determines a position and orientation of the scanner 104-1 in a coordinate system, e.g., the coordinate system 228 in FIG. 2. The IMU can include a combination of accelerometers, gyroscopes, and magnetometers, and generate positional data including data representing six degrees of freedom (6DOF), such as yaw, pitch, and roll angles in the coordinate system. Typically, 6DOF refers to the freedom of movement of a body in three-dimensional space. For example, the body is free to change position as forward/backward (surge), up/down (heave), left/right (sway) translation in three perpendicular axes, combined with changes in orientation through rotation about three perpendicular axes, often termed yaw (normal axis), pitch (transverse axis), and roll (longitudinal axis). Additionally or alternatively, the ultrasound system can include a camera and fiducial markers on the scanner 104-1 (not shown in FIG. 2) to determine the positional data for the ultrasound scanner 104-1. In one example, the system generates, based on the positional data, a trigger signal as described above. For some embodiments the positional data can indicate that the scanner 104 is within a threshold distance of the patient, and the trigger signal can be used by the ultrasound system to enable one or more machine-learned models, e.g., to generate a health status report for a port, as described above.

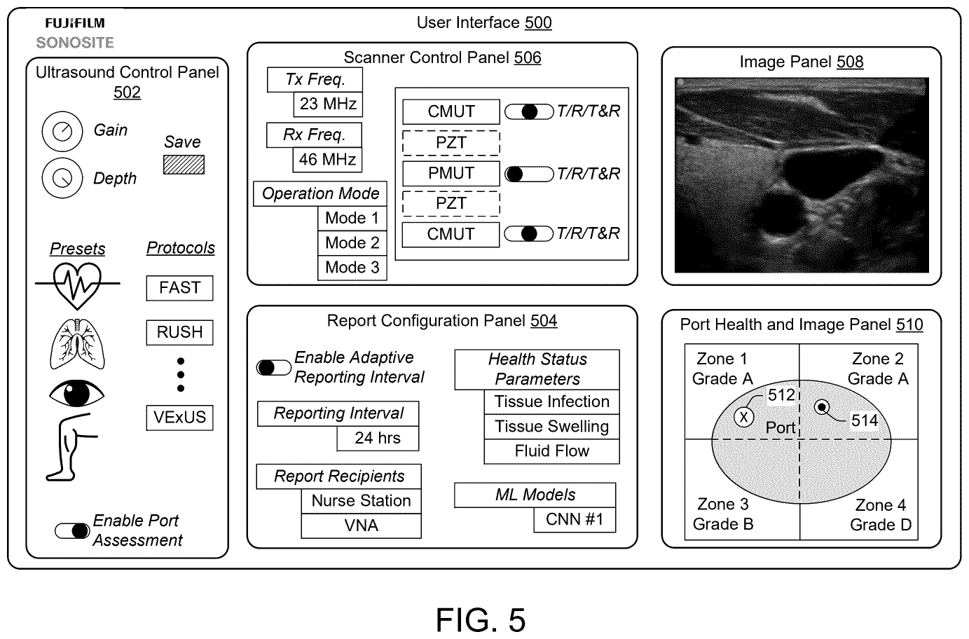

A trigger signal generated by the system, e.g., due to pressure data and/or positional data as described above, can be used to expedite the workflow of the system. In some embodiments, responsive to a trigger signal, the system automatically requests a patient history, such as from a medical archiver (e.g., a VNA). The patient history can include previously-generated port health status reports for the patient, which can be displayed by the system for comparison against a current port health status report. In some embodiments, responsive to a trigger signal, the system causes a port health and image panel (e.g., the port health and image panel 510 in FIG. 5) to be displayed by a computing device. In another example, the system can, responsive to a trigger signal, enable one or more arrays of a scanner (e.g., the scanner 104), according to an operation mode of the system. Example operations modes are described below with respect to Table 1.

The scanner 104 also includes example scanners 104-2 and 104-3 that are coupled to the ultrasound machine via the coupling 210, e.g., via a wireless connection. The scanners 104-2 and 104-3 are examples of wearable ultrasound scanners that can be patient worn, such as patches that can be placed over a port that has been installed in a patient. The scanners 104-2 and 104-3 can be used during an ultrasound examination, e.g., for testing, diagnostic, therapeutic, or procedural reasons. Additionally or alternatively, the scanners 104-2 and 104-3 can be placed on a patient for longer term monitoring, e.g., days, weeks, or months, to continuously (e.g., periodically) monitor the health of a port and generate a health status report. Hence, the scanners 104-2 and 104-3 can be used remotely by the patient, so that the patient can forego visits to a care facility to determine a health status of a port. In some embodiments, in the case of a patch with wearable ultrasound scanners, the patch (and/or port) can include one or more communication interfaces (electronics) to send data from the wearable ultrasound scanners 104-2 and 104-3 to a remote location (e.g., a network) to provide the health status of the port. The sending of such data can be automatic upon uses of the scanner or at certain times (e.g., predetermined times, regular intervals, etc.). The communication interfaces can also be used to receive data from another source (e.g., medical machine, medical vital sign capture device, medicine receptacle, etc.) which can be combined and sent with the data from scanners 104-2 and 104-3. The health status report can include a prediction of when the port will fail. Hence, a patient can get the port repaired or replaced prior to the port failure, so that the patient does not need to miss or delay a procedure that requires the port, such as chemotherapy treatment or blood transfusion.

In some embodiments, the time intervals (e.g., periodic intervals) that the system generates a health status report are generated adaptively by the system. For example, the time intervals can be based on a previously-determined health of the port from the scanners 104-2 and 104-3. For instance, if the system determines a health status of the port that includes severe tissue swelling, infection, etc., and/or an expected (e.g., predicted) time of port failure that is short (e.g., within a few days), the system can generate new health statuses for the port more frequently than if no swelling or infection is detected, or if the expected time to failure for the port is long (e.g., in two months). In some embodiments, the scanners 104-2 and 104-3 can generate the health status reports without intervention by the patient, and automatically and without user intervention send a health status of the port to another party, such as a nurse station, doctor, medical archiver, etc.

The scanner 104 also includes the example scanner 104-4, which is configured for handheld operation like the scanner 104-1 and includes removably attachable electronics 230 and removably attachable ultrasound arrays 232. The removably attachable electronics 230 can be removed from the body of the scanner 104-4 and re-attached. Any suitable attachment mechanism can be used for removal and attachment of the removably attachable electronics 230 and the removably attachable ultrasound arrays 232 from/to the scanner 104-4, such as rails, dovetails, clamps, fasteners, gaskets, O-rings, etc. In some embodiments, the removably attachable electronics 230 and the removably attachable ultrasound arrays 232 can be attached to the body of the scanner 104-4 and maintain an IPX7-rated seal.

Examples of the removably attachable electronics 230 include removably attachable electronics 230-1 and 230-2. The removably attachable electronics 230-1 include circuitry so that the scanner 104-4 can communicate with an ultrasound machine, e.g., the ultrasound machine 102, over a wireless communication link 234. The removably attachable electronics 230-2 include circuitry so that the scanner 104-4 can communicate with an ultrasound machine, e.g., the ultrasound machine 102, over a wired communication link 236, such as via one or more cables. Hence, the scanner 104-4 can be reconfigured for use with different ultrasound machines, including ones that support wired coupling to the scanner 104-4 and others that support wireless coupling to the scanner 104-4.

Examples of the removably attachable ultrasound arrays 232 include removably attachable arrays 232-1 and 232-2. The removably attachable arrays 232-1 and 232-2 can include multi-array transducer assemblies (e.g., as discussed below with respect to FIGS. 9-14). Accordingly, the removably attachable arrays 232-1 and 232-2 can include multi-array transducer assemblies that include any combination of piezoelectric micromachined ultrasonic transducer (PMUT) array elements, lead zirconate titanate (PZT) array elements, and capacitive micromachined ultrasonic transducer (CMUT) array elements. In some embodiments, the removably attachable array 232-1 includes at least one array with PZT array elements and the removably attachable array 232-2 includes at least one array with CMUT array elements. In another example, the removably attachable array 232-1 includes at least one array with PMUT array elements and the removably attachable array 232-2 includes at least one array with CMUT array elements. In still another example, at least one of the removably attachable arrays 232-1 and 232-2 includes a first array with array elements selected from the group consisting of PZT, PMUT, and CMUT array elements, and a second array with additional array elements selected from the group consisting of PZT, PMUT, and CMUT array elements. The elements of the first array can be of a different type than the elements of the second array (e.g., the first array can include PMUT elements and the second array can include CMUT elements). Alternatively, the elements of the first array can be of a same type as the elements of the second array (e.g., the first array and the second array can both include PMUT elements). Hence, the user may not need to carry an assortment of different scanners, but instead carry the scanner 104-4 with removably attachable ultrasound arrays 232 that can be used for different types of examinations.

FIG. 3 illustrates some embodiments of a system in an environment 300 for determining port health with ultrasound. The environment 300 includes a patient 302 who is wearing a wearable ultrasound scanner 304. The wearable ultrasound scanner 304 is an example of the scanners 104-2 and 104-3 in FIG. 2. The wearable ultrasound scanner 304 can be placed over a port installed on the patient 302 (e.g., the port having reservoir 314 and line 316, and the port illustrated at inlay 328).

The wearable ultrasound scanner 304 includes circuitry 306, as is illustrated in profile in FIG. 3. The circuitry 306 can include any suitable electronics to process and/or display ultrasound data generated by the wearable ultrasound scanner 304, and communicate data to another device, such as the ultrasound machine 102 or any of the computing devices illustrated in FIG. 3. For example, the circuitry 306 can include the system electronics 216, the memory 110, the ultrasound control subsystem 218, the ultrasound imaging subsystem 220, the processors 106, the transceiver 112, the display device 108, and the like. The circuitry 306 can communicate over the network 318, e.g., via Bluetooth™, Wi-Fi™, etc. In some embodiments, the network 318 includes a network maintained by a care facility, e.g., an intranet. Additionally or alternatively, the network 318 can include the Internet.

The wearable ultrasound scanner 304 also includes one or more transducer arrays 308. The transducer arrays 308 can include any suitable type of transducer array, including one or more transducer arrays that include one or more elements selected from the group consisting of piezoelectric micromachined ultrasonic transducer (PMUT) array elements, lead zirconate titanate (PZT) array elements, and capacitive micromachined ultrasonic transducer (CMUT) array elements. In an example, the wearable ultrasound scanner 304 is a multi-array ultrasound scanner that includes one or more transducer arrays having PZT array elements and one or more additional transducer arrays having PMUT array elements. In another example, the wearable ultrasound scanner 304 is a multi-array ultrasound scanner that includes one or more transducer arrays having PZT array elements and one or more additional transducer arrays having CMUT array elements. In still another example, the wearable ultrasound scanner 304 is a multi-array ultrasound scanner that includes one or more transducer arrays having PMUT array elements and one or more additional transducer arrays having CMUT array elements.

In some embodiments, the wearable ultrasound scanner 304 is a multi-array ultrasound scanner that includes multiple transducer arrays having PZT array elements. In some embodiments, the wearable ultrasound scanner 304 is a multi-array ultrasound scanner that includes multiple transducer arrays having PMUT array elements. In some embodiments, the wearable ultrasound scanner 304 is a multi-array ultrasound scanner that includes multiple transducer arrays having CMUT array elements.

The wearable ultrasound scanner 304 can include a lens (not shown in FIG. 3 for clarity). The lens can be placed over the transducer arrays 308 and facing the patient. In some embodiments, the wearable ultrasound scanner 304 also includes a coupling agent 310 that couples ultrasound from the transducer arrays 308 to the patient anatomy and the port having reservoir 314 and line 316, as well as reflections of the ultrasound from the patient anatomy and the port back to the transducer arrays 308. The coupling agent 310 can be encapsulated in a packet (e.g., a gel pack). In some embodiments, the gel pack is affixed to a tegaderm pad or patch. In some embodiments, when determining port health with ultrasound, the packet can be affixed to the transducer arrays 308 or a lens covering the transducer arrays. The packet can be held in place with pressure, a pocket or recess to hold the packet, a clip or stand to retain the packet, or combinations thereof. In some embodiments, the coupling agent 310 can lack a bounding container, like a packet, and instead include a gel or paste.

The wearable ultrasound scanner 304 is placed against the patient skin 312, over the port having reservoir 314 and line 316. A photograph of an example of the port is illustrated in the inlay 328 in FIG. 3. The port's reservoir 314 can include one or more access points for a needle, and the port's line 316 can be inserted into a blood vessel of the patient 302. Hence, fluid can be drawn from the blood vessel via the port, and/or inserted into the blood vessel via the port. The wearable ultrasound scanner 304 can be affixed to the patient skin 312 via any suitable mechanism, including glue, tape, a tegaderm patch, a bandage wrapped around the patient, etc.

The wearable ultrasound scanner 304 can generate ultrasound and receive reflections of the ultrasound from the patient's 302 anatomy and the port's reservoir 314 and line 316. Based on the ultrasound reflections, the wearable ultrasound scanner 304 can determine a health status of the port. For example, the wearable ultrasound scanner 304 can determine, based on the ultrasound data, one or more parameters, including an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of the fluid or the additional fluid. In some embodiments, the circuitry 306 implements one or more machine-learned models to determine these parameters. The circuitry 306 can also implement a machine-learned model (e.g., a convolutional neural network) to generate, based on one or more of these parameters, a health status report for the port. The health status report can include an estimated (e.g., predicted) time to failure for the port. Additionally or alternatively, the health status report can include a grade or score for the health of the port, such as a letter grade in the scale of “A” to “F”, or a number score in the scale of one to ten. Additionally or alternatively, the health status report can include an indication that a port has already failed and should not be used for fluid insertion or removal. In some embodiments, the wearable ultrasound scanner 304 can generate a recommendation, such as a recommended insertion point of the needle, a recommended date for a follow-up examination, etc.

The wearable ultrasound scanner 304 can communicate any suitable data, such as the health status report of a port, over the network 318 to a computing device coupled to the network 318, including the computing device 320 (e.g., a smart phone or tablet, such as a doctor's personal computing device), a device at a nurse station 322, a medical archiver 324, a server 326, and the ultrasound machine 102. By communicating the health status report to the nurse station 322, care facility staff can be kept up to date on the health of the patient's port and make appropriate scheduling decisions. For instance, the staff can adjust the timing of a scheduled treatment so that it is completed before the health of the port deteriorates below a threshold level or reschedule a scheduled treatment until after the port has been repaired or replaced.

The server 326 can include one or more server devices, such as a server system maintained by a care facility. A clinician may have access to the server 326 to review a health status report for a patient. The medical archiver 324 can maintain patient data and update the patient's 302 records with a health status report of a port provided by the wearable ultrasound scanner 304. The medical archiver 324 can also provide previous patient data for comparison to current patient data. For instance, a clinician or the patient 302 can compare a current health status report generated with the wearable ultrasound scanner 304 to previous health status reports generated with the wearable ultrasound scanner 304 or another ultrasound scanner.

In some embodiments, a computing device (e.g., the computing device 320) can display guidance to a user to help place the wearable ultrasound scanner 304 at a location and orientation on the patient 302 to image the port having the reservoir 314 and the line 316 to generate a health status report of the port. For instance, the computing device 320 can display directional arrows, angles, etc. to align the wearable ultrasound scanner 304 with the port so that a desired view is achieved in an ultrasound image. In some embodiments, the computing device 320 displays an ultrasound image having a view that the user can match by maneuvering the wearable ultrasound scanner 304 for placement on the patient 302. The wearable ultrasound scanner 304 can include any suitable glue, tape, binder, etc. to anchor the wearable ultrasound scanner 304 to the patient 302.

In some embodiments, the wearable ultrasound scanner 304 can be used for other applications than determining port health with ultrasound. For example, in some embodiments, the wearable ultrasound scanner 304 is used for monitoring fluid status in a patient. In another example, in some other embodiments, the wearable ultrasound scanner 304 can be used for cardiac monitoring, such as to monitor the size of a patient's heart, or a left ventricle parameter, such as ejection fraction. In another example, in yet some other embodiments, the wearable ultrasound scanner 304 can be used for respiratory monitoring, such as for determining lung sliding or b-line assessment. Monitoring can be done during a procedure, after a procedure, or before a procedure, including for diagnostic or therapeutic reasons, such as for pain management, neuropathy treatment, or to break up calcium deposits. In some embodiments, the wearable ultrasound scanner 304 is applied to a patient's temporal region for monitoring intercranial pressure and/or optic nerve assessment. In some embodiments, the system includes a vest that includes multiple wearable ultrasound scanners, such as multiple wearable ultrasound scanners 403. The vest can be worn by a patient and used for monitoring abdominal, chest, cardiac, and other anatomies for diagnostic, therapeutic, or procedural reasons.

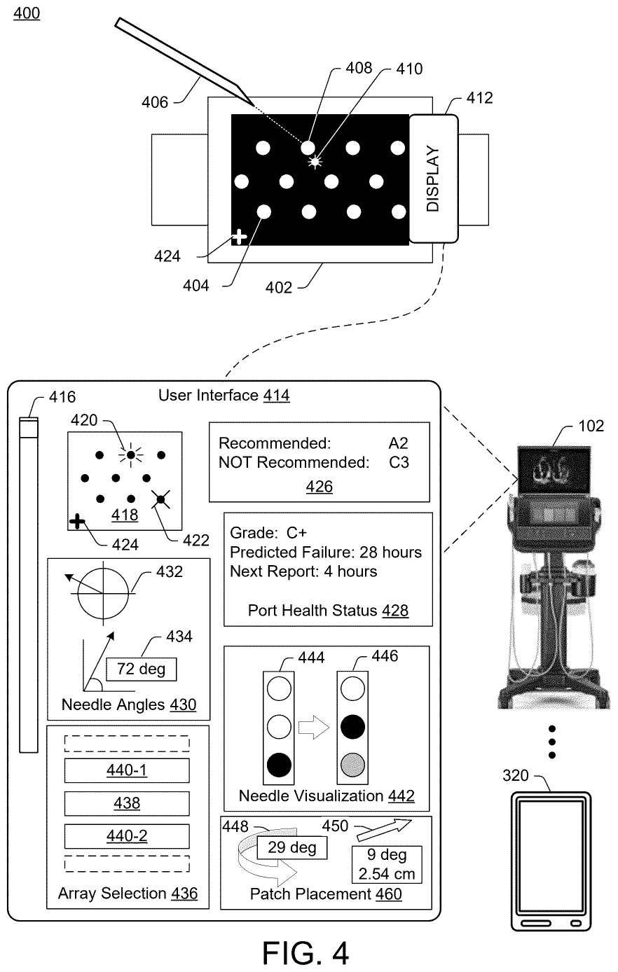

FIG. 4 illustrates some embodiments of a system 400 for determining port health with ultrasound. The system 400 includes a wearable ultrasound scanner 402, which is an example of the wearable ultrasound scanner 304 in FIG. 3 and the wearable scanners 104-2 and 104-3 in FIG. 2. In some embodiments, the wearable ultrasound scanner 402 includes a patch that can be affixed to a patient over a port installed in the patient. In some embodiments, the wearable ultrasound scanner 402 includes multiple access holes 404 through which an interventional instrument 406, such as a needle, can be inserted into a port to supply fluid into the patient and/or remove fluid from the patient. For clarity, only one of the holes is designated as 404.

In some embodiments, the system 400 generates a recommendation that includes a recommended hole 408 for insertion of the interventional instrument 406 and indicates the recommended hole 408 by illuminating one or more light sources 410 that are proximate to the recommended hole 408. In some embodiments, the system 400 implements one or more machine-learned models to generate the recommendation that includes the recommended hole 408. For instance, the machine-learned model can include a convolutional neural network that can process one or more parameters determined based on ultrasound generated by the wearable ultrasound scanner 402. Example parameters include an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of fluid input to, or removed from, the patient. One or more of these parameters can be concatenated into an input vector that is processed by the convolutional neural network. Alternatively, some of these parameters can be concatenated into an input vector that is processed by layers of the convolutional neural network to generate a feature vector. The others of these parameters can be provided as conditional inputs to the convolutional neural network and concatenated with the feature vector that the convolutional neural network generates at the output of said layers of the convolutional neural network. The resulting concatenated vector can be processed by additional layers of the convolutional neural network and used to generate the recommended hole 408. In this way, the convolutional neural network can process some of the parameters at the top layer, and others of the parameters at intermediate layers.

Additionally or alternatively, the system 400 can implement one or more machine-learned models to generate a health status report of the port. In some embodiments, the system can then generate, based on the health status report, the recommended hole 408 for insertion of the interventional instrument 406, e.g., a needle. By recommending one of the holes for insertion of the needle, the system can prevent overuse of an area of the port and prolong the port's time to failure. By prolonging the time to failure for the port, the patient may be able to forego the installation or repair of the port, thus reducing or eliminating the need to reschedule or delay procedures that require port access. Further, the system can instruct the user to avoid areas of the port that are proximate to infected or inflamed tissue, thus reducing pain and discomfort for the patient. In some embodiments, the system can determine the recommended hole 408 for insertion so as to avoid these areas. Additionally or alternatively, the system can determine the recommended hole 408 for insertion so as to avoid a location on the port that was used for one or more previous needle insertions.

Note that the recommendations from monitoring with ultrasound are not limited to provided recommended holes for insertion of a needle or other instrument. For example, the recommendations can include a recommendation as to the substance to inject into a port. Such a substance can be used to address inflammation in the tissue around the port. As another example, the recommendations can include whether to attach a certain type of pump to the port (e.g., pain pump, infusion pump, chemotherapy pump, etc.).

The wearable ultrasound scanner 402 can also include a display 412. In some embodiments, the display 412 is removably attached to the wearable ultrasound scanner 402. Hence, the wearable ultrasound scanner 402 can be disposable, and the display device can be sterilized for reuse with another wearable ultrasound scanner. In some embodiments, the display 412 can be removably attached to the wearable ultrasound scanner 402 via any suitable mechanism, such as hook-and-loop fasteners, molded plastic parts that clasp the display 412, magnets, etc.

The display 412 can display a user interface 414. Additionally or alternatively, the user interface 414 can be displayed by another device, such as the ultrasound machine 102 and/or the computing device 320. In some embodiments, the user interface 414 includes a slider 416 to enable a user to scroll through content displayed on the user interface 414. In some embodiments, the user interface 414 includes a visual representation 418 that represents the holes 404 of the wearable ultrasound scanner 402. In the example in FIG. 4, the visual representation 418 includes a pattern representing the holes 404. The recommended hole 408 for insertion is indicated by a light source 420 on the visual representation 418. The light source 420 on the user interface 414 can indicate the recommended hole 408 for insertion in a similar manner as the light source 410 on the wearable ultrasound scanner 402, such as by blinking on and off, blinking in a pattern of on and off, changing color, changing intensity, etc.

In some embodiments, the visual representation 418 also includes a recommendation 422 that indicates one of the insertion holes to avoid (e.g., not to use) for needle insertion. The system 400 can determine the recommendation 422 for an insertion hole to avoid based on a history of needle insertions. For example, if one of the holes 404 was used for needle insertion in a previous treatment, then the system can determine not to use that hole for a needle insertion for a current treatment. Thus, the system can prevent the overuse of an insertion hole to prolong the life of the port and reduce the impact on the patient's tissue. In some embodiments, the visual representation 418 displays a registration mark 424 to align the orientation of the holes of the visual representation 418 with the holes of the wearable ultrasound scanner 402, which can also include the registration mark 424 (or a similar, matching registration mark).

In some embodiments, the user interface 414 also includes a panel 426 that can indicate recommended holes for insertion (e.g., the hole indicated by the light source 420) and holes not recommended for insertion (e.g., the hole indicated by the recommendation 422). In the example in FIG. 4, the panel 426 displays text that indicates the holes to use and not to use. The text indicates a row with a letter and a column with a number. For instance, “A2” refers to the hole 408 and “C3” refers to the hole indicated by the recommendation 422.

In some embodiments, the user interface 414 also includes a port health status panel 428 that can include any data generated by the system 400 as part of a health status report for a port. For instance, the port health status panel 428 in FIG. 4 includes a grade of C+ for the port (e.g., on a scale of A-F), and a predicted time to failure for the port of 28 hours. The port health status panel 428 also includes an indication that the system 400 will generate a next health status report for the port in 4 hours from the present time.

In some embodiments, the user interface 414 also includes a needle angles panel 430 that can include any suitable data to indicate a recommended orientation for a needle that is inserted into the port, e.g., by inserting the needle into the recommended hole 408. In the example in FIG. 4, the needle angles panel 430 includes a visual representation 432 that indicates a recommended angle of the needle (e.g., the interventional instrument 406) in the horizontal plane (e.g., coplanar with the wearable ultrasound scanner 402). The angle can be relative to a coordinate system, such as axes that represent edges of the wearable ultrasound scanner 402, or the X-Y axes of the coordinate system 228 in FIG. 2. The needle angles panel 430 includes a visual representation 434 that indicates a recommended angle of the needle (e.g., the interventional instrument 406) in a vertical plane, e.g., a steepness angle. The angle can be in a Z-dimension of the coordinate system 228 in FIG. 2. In FIG. 4, the visual representation 434 includes both a graphic of the angle and text indicating that the recommended angle is 72 degrees.

In some embodiments, the user interface 414 also includes an array selection panel 436 that can include any suitable input or control to configure one or more arrays of the wearable ultrasound scanner 402. The array selection panel 436 in FIG. 4 includes a visual representation of five arrays of a multi-array transducer included in the wearable ultrasound scanner 402. A user can enable one or more of the five arrays by tapping on the visual representation for an array. For instance, the center array 438 is indicated as enabled, and two arrays 440-1 and 440-2 immediately adjacent to the center array 438 are also indicated as enabled (evidenced by the solid boxes for these arrays). The two outer-most arrays are indicated as not enabled, evidenced by the dashed boxes for these arrays.

In some embodiments, the user interface 414 also includes a needle visualization panel 442 that can include any suitable visual aids to visualize the needle 406, including the tip of the needle 406. In the example in FIG. 4, the needle visualization panel 442 includes a first visual representation 444 and a second visual representation 446 useful for out-of-plane needle visualization. The visual representations 444 and 446 each include three circles. The fill content of the circles indicates if the needle tip has been detected by the arrays, 438, 440-1, and 440-2. For instance, the top circle of the visual representations 444 and 446 corresponds to the array 440-1, the middle circle of the visual representations 444 and 446 corresponds to the array 438, and the bottom circle of the visual representations 444 and 446 corresponds to the array 440-2. Black fill content of a circle indicates that the tip of the needle 406 is currently detected by the array corresponding to the circle. Grey fill content of a circle indicates that the tip of the needle 406 was previously detected by the array corresponding to the circle and that the shaft of the needle 406 is currently detected by the circle. White fill content of a circle indicates that the needle 406 has not been detected by the array corresponding to the circle. In the example in FIG. 4, during operation the needle visualization panel 442 can first display the visual representation 444 to indicate that the tip of the needle 406 is detected by the array 440-2. When the tip of the needle passes through the imaging plane of the array 440-2, and into the imaging plane of the array 338, the needle visualization panel 442 can then display the visual representation 446. Accordingly, the needle visualization panel 442 can provide visual assistance to indicate a current position of the tip of the needle 406, as well as a trajectory of the needle 406.

In some embodiments, the user interface 414 also includes a patch placement panel 460 that can include any suitable visual aids to guide a user to place a wearable ultrasound scanner (e.g., the wearable ultrasound scanner 402) on a patient. In the example in FIG. 4, the patch placement panel 460 includes a first visual representation 448 that indicates to rotate the patch counter-clockwise by 29 degrees, and a second visual representation 450 that indicates to translate (e.g., move) the patch by 2.54 cm in a direction corresponding to a 9 degree vector. The system can generate the visual aids and guidance in any suitable way. In some embodiments, the system implements one or more machine-learned models that process ultrasound images generated by the wearable ultrasound scanner 402 as it is being placed on the patient. The machine-learned model can generate the guidance instructions as images, text, icons, combinations thereof, and the like. In some embodiments, the system uses registration marks on the patient, such as temporary tattoos or fiducial markers, to generate the guidance instructions. For example, the system can include one or more cameras that can image the registration marks on the patient and the registration mark 424 on the wearable ultrasound scanner 402. A machine-learned model can then generate the guidance instructions by processing the images from the cameras. Additionally or alternatively, the machine-learned model can process ultrasound images generated by the wearable ultrasound scanner 402 during its placement to generate the guidance instructions. For instance, a first image can be provided as an input to a top layer of a convolutional neural network (CNN) and a second image can be provided as a secondary input to a subsequent layer of the CNN and concatenated with a feature vector generated at the output of the subsequent layer based on the first image. Based on these images input to the CNN, it can generate the guidance instructions.

FIG. 5 illustrates some embodiments of a user interface 500 of a system for determining port health with ultrasound. The user interface can be displayed on any suitable computing device of the system for determining port health with ultrasound, including one or more of the ultrasound machine 102, the computing device 320, the server 326, and the wearable ultrasound device 402. In some embodiments, the interface 500 includes an ultrasound control panel 502, a report configuration panel 504, a scanner control panel 506, an image panel 508, and a port health and image panel 510.

In some embodiments, the ultrasound control panel 502 can include any suitable controls for configuring an ultrasound system for determining port health with ultrasound. In the example in FIG. 5, the ultrasound control panel 502 includes ultrasound controls for adjusting gain and depth, saving an image, and selecting examination presets. The examination presets are represented by selectable icons for a cardiac examination, a respiratory examination, an ocular examination, and a muscular-skeletal examination. These examination presets, when selected, can configure the ultrasound machine with predetermined values of gain and depth, and other imaging parameters (e.g., beamformer settings, filter coefficients, amplitude settings, etc.). The ultrasound control panel 502 also includes controls for selecting ultrasound protocols, including a Focused Assessment with Sonography for Trauma (FAST) protocol, a Rapid Ultrasound for Shock and Hypotension (RUSH) protocol, and a Venous Congestion Evaluation using Ultrasound (VExUS) protocol. A user can select one of these example protocols, and in response, the system can configure itself for an examination in accordance with the protocol, including to display a protocol panel in the user interface 500 (not shown for clarity) with guided steps needed to complete the selected protocol. The ultrasound control panel 502 also includes a selection (e.g., an electronic rocker switch) to enable port assessment.

Responsive to the selection to enable port assessment (e.g., via the rocker switch in the ultrasound control panel 502), the user interface 500 displays the report configuration panel 504. The report configuration panel 504 can display any suitable option, control, or setting to configure determining port health with ultrasound. In the example in FIG. 5, the report configuration panel 504 includes an electronic rocker switch to enable an adaptive reporting interval. When selected, the system can be adaptive when determining a time (e.g., time period) to generate and communicate a health status report of a port adaptively, e.g., based on the health status of the port. For instance, if the system determines a port is likely to fail within the next three days, the system can increase the rate at which the system generates and communicates a health status of the port, compared to if the system determines the port is estimated to fail in three months. As indicated in the example in FIG. 5, the adaptive reporting interval is disabled, and the reporting interval is set via a drop-down menu to 24 hours. Hence, in this configuration, the system can generate, based on ultrasound from a wearable ultrasound scanner, a health status of a port covered by the wearable ultrasound scanner every 24 hours, and communicate the health status of the port to a computing device, such as an ultrasound machine, clinician's personal computing device, nursing station, medical archiver, and the like. In the example in FIG. 5, a user has configured the system to send the report to a nurse's station and a vendor neutral archive (VNA), as evidenced by the drop-down menu selections in the report configuration panel 504.

The report configuration panel 504 also includes options for selection of the parameters used by the system to generate the health status of the port. In the example in FIG. 5, the system is configured to generate the health status report of the port based on tissue infection, tissue swelling, and fluid flow, and use a machine-learned model named CNN #1, as indicated by the drop-down menu selections in the report configuration panel 504. In this configuration, the system can enable the machine-learned model CNN #1 to process one or more ultrasound images generated with a wearable ultrasound scanner and generate the health status report by determining from the images measures of tissue infection and tissue swelling for tissue proximate to the port, and fluid flow for fluid in the port. In some embodiments, the report configuration panel 504 includes an option for automatically determining the health status parameters used for determining the health status of the port, and selecting the machine-learned model that generates the health status report (not shown in FIG. 5 for clarity).

In some embodiments, the user interface 500 also includes the scanner control panel 506 that includes controls and selections for configuring one or more arrays of a transducer assembly of an ultrasound scanner, including a wearable ultrasound scanner and a handheld ultrasound scanner. In some embodiments, the scanner control panel 506 includes options to select transmit and receive frequencies for one or more arrays of an ultrasound scanner. In the example in FIG. 5, a transmit frequency is set via a drop-down menu to 23 MHz, and a receive frequency is set via a drop-down menu to 46 MHz. In some embodiments, the scanner control panel 506 also includes options for enabling and configuring one or more arrays of a multi-array transducer. In the example in FIG. 5, the multi-array scanner includes five arrays (or sub-arrays), including a center array comprised of PMUT array elements, two adjacent arrays comprised of PZT array elements, and two outer arrays comprised of CMUT array elements. As indicated by the dashed lines, the PZT arrays are disabled, and as indicated by the solid lines, the PMUT and CMUT arrays are enabled. In some embodiments, a user can enable and disable an array by touching or tapping on the visual representation for the array. The scanner control panel 506 also includes options (e.g., three-position electronic rocker switches) to configure the enabled arrays for transmission, reception, or both transmission and reception. In the example in FIG. 5, the PMUT array (e.g., the center array) is configured to transmit, and the CMUT arrays (e.g., the outer arrays) are configured to receive. In some embodiments, the PMUT arrays have better transmit sensitivity (in terms of power efficiency) than the CMUT arrays, while the CMUT arrays have better receive sensitivity (in terms of signal strength) than the PMUT arrays.

In some embodiments, the scanner control panel 506 also includes a drop-down menu to enable the ultrasound scanner according to an operation mode. Example operation modes are described below with respect to Table 1, and include Mode 1 which can enable at least one array of an ultrasound scanner as a linear array and at least one additional array of the ultrasound scanner as a phased array, Mode 2 which can enable the arrays for broadband tissue harmonic imaging (broadband THI), and Mode 3 which can enable the arrays for full-aperture broadband THI operation.

In some embodiments, the user interface 500 also includes the image panel 508 for displaying any suitable type and number of ultrasound images. In the example in FIG. 5, the image panel 508 displays a B-mode image that includes blood vessels. A port can be inserted into the patient, and one end of the port can be inserted into a blood vessel of the image to insert and/or draw fluid.

In some embodiments, the user interface 500 also includes the port health and image panel 510 for indicating a health status of a port. The port health and image panel 510 includes a visual representation of a port, in this case an ellipse. The visual representation can include any suitable type of visual representation, such as an illustration, a photograph, an animation, an ultrasound image, etc. The port and surrounding tissue of the port are broken up into four zones 1-4, and the system assigns each of the four zones 1-4 a grade as part of generating a health status report of a port. Zones 1 and 2 are assigned an A grade, zone 3 is assigned a B grade, and zone 4 is assigned a D grade. Further, as an example, the system illustrates the previous needle insertion point 512 in zone 1, e.g., from a previous procedure, treatment, or examination. To avoid overuse of the port and tissue near the previous needle insertion point 512 in zone 1, and based on the health status of the port generated by the system (e.g., an A grade in zone 2), in some embodiments, the system recommends an insertion point 514 for the port in zone 2. In some embodiments, a wearable ultrasound scanner includes insertion holes (e.g., the access holes 404 in FIG. 4), and the system recommends one of the access holes for insertion of a needle, including guidance for needle orientation, so that the needle tip hits the insertion point 514.

In some embodiments, the user interface 500 includes a panel or other area that shows one or more patches that are available for monitoring. For example, the user interface 500 can show patches 1-5. In some embodiments, the user is able to select one (or more) of the patches on the user interface 500, which then causes data or other feedback from the selected patch(es) to be displayed in the user interface 500.

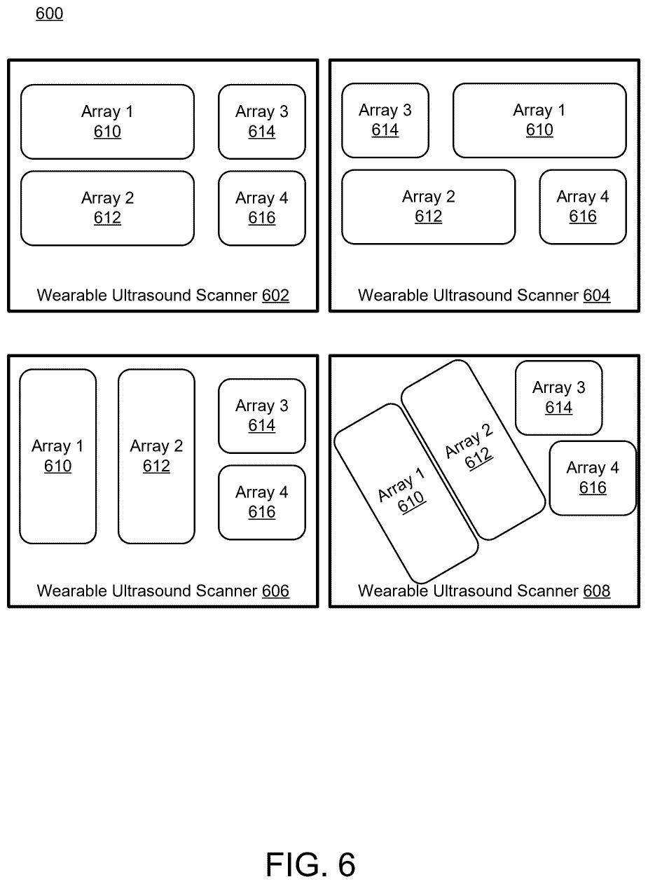

FIG. 6 illustrates some embodiments of configurations 600 of a reconfigurable wearable ultrasound scanner for determining port health with ultrasound. The configurations 600 include reconfigurable wearable ultrasound scanners 602-608, each of which include transducer arrays 610-616. In some embodiments, the transducer arrays 610-616 can include any suitable number of arrays. In some embodiments, one or more of the transducer arrays 610-616 include a multi-array transducer. A multi-array transducer can include any combination of PZT, PMUT, and CMUT arrays, such as is described below with respect to FIGS. 9-14. The transducer arrays 610-616 can be removably attached to a wearable ultrasound scanner, such as a patch-based wearable ultrasound scanner as previously described (e.g., the wearable ultrasound scanners 104-2, 104-2, 304, and 402). For example, the transducer arrays 610-616 can be removed from a wearable ultrasound scanner, repositioned or reoriented, and again attached to the wearable ultrasound scanner for use. The reconfigurable wearable ultrasound scanners 602-608 in FIG. 6 illustrate four examples in which the transducer arrays 610-616 are arranged in different orientations. The transducer arrays 610-616 can be removably attached to a wearable ultrasound scanner via any suitable mechanism, such as hook-and-loop fasteners, molded plastic parts that clasp the transducer arrays 610-616, magnets, etc.

By reconfiguring one or more of the transducer arrays 610-616 on the wearable ultrasound scanner, the wearable ultrasound scanner can remain on the patient and the arrays can be rotated and/or translated on the wearable ultrasound scanner. Hence, the wearable ultrasound scanner does not need to be removed from the patient to configure the system to obtain ultrasound images from different perspectives, or images of different anatomies, or images with different types of arrays. Further, one or more of the transducer arrays 610-616 can be removed from the wearable ultrasound scanner when it is not needed. For instance, suppose a certain procedure uses a CMUT transducer array and the procedure will not be performed for three months. During the three months, one or more PMUT transducer arrays can be attached to the wearable ultrasound scanner and used for port monitoring. The CMUT transducer array can then be attached to the wearable ultrasound scanner at the expiration of the three months to perform the procedure. In some embodiments, one or more of the transducer arrays 610-616 can be removed and replaced based on the type of treatment. For instance, a first array can be selected and installed for diagnostic purposes on the wearable ultrasound scanner. The first array can later be removed and replaced with a second array for therapeutic purposes. Hence, the transducer arrays 610-616 can be removably attached and replaced on the wearable ultrasound scanner to track the patient's treatment progress, without requiring removal of wearable ultrasound scanner from the patient.

In some embodiments, the transducer arrays can be positioned in a particular orientation. For example, the transducer arrays can be positioned to affect imaging that is to be performed, such as, biplane imaging. Biplane imaging can be useful when inserting a needle by providing an indication that the needle is in plane.

In some embodiments, patches having one or more transducer arrays can be combined. Such a combination can results in the same configurations of transducer arrays in FIG. 6 or other configurations. In some embodiments, the patches are combined by interleaving ultrasound data generated by different patches and processing the ultrasound data by a processing system in a joint manner, e.g., by treating the different patches as nodes in a synthetic aperture sensor system.

FIG. 7 illustrates an environment 700 with an example ultrasound scanner used for in-plane needle insertion. Referring to FIG. 7, the ultrasound scanner includes a light source 702. An example of the light source 702 includes a microelectromechanical systems (MEMS) emitter (e.g., a MEMS laser). The light source 702 projects a light onto the patient skin to indicate an insertion point 704 and trace a blood vessel 706. The ultrasound scanner can include a multi-array transducer, as is described below with respect to FIGS. 9-14. In some embodiments, the light source 702 can indicate a current position of the tip of the needle based on which array of the multi-array ultrasound scanner detects the needle tip in its imaging plane. For instance, the multi-array ultrasound scanner can include three arrays: a left array, a center array, and a right array. When the needle is in the imaging plane of the center array, the light source 702 can emit a green light. However, if the needle moves to the imaging plane of the left array or the right array, the light source 702 can change the color of the light emitted to red. In some embodiments, the color of the light emitted by the light source 702 corresponds to the array in which the needle is in plane. For instance, green can indicate that the needle is in the imaging plane of the center array, red can indicate that the needle is in the imaging plane of the left array, and orange can indicate that the needle is in the imaging plane of the right array. Additionally or alternatively, the light source 702 can change its intensity and/or blinking pattern to indicate which array's imaging plane corresponds to the current needle tip position. In this way, the light source 702 can guide the user to maintain the needle in-plane.

In some embodiments, the light source 702 includes multiple light sources spatially separated on the ultrasound scanner, such as side-by-side (not shown in FIG. 7 for clarity). Each of the arrays can correspond to one of the light sources. When the needle is in the imaging plane of one of the arrays, the system can cause the light source that corresponds to that array to project light. Hence, the user can determine if they are inserting the needle straight with respect to the lateral dimension of the ultrasound scanner by inspection of which light source is projecting light, and/or the color of the light, and/or the blinking pattern of the light.

FIG. 8 illustrates an environment 800 with an example ultrasound scanner used for out-of-plane needle insertion. The ultrasound scanner includes a light source 802. An example of the light source 802 includes a MEMS emitter (e.g., a MEMS laser). The light source 802 projects a light onto the patient skin to indicate an insertion point 804 and trace a blood vessel 806. The ultrasound scanner can include a multi-array transducer, as is described below with respect to FIGS. 9-14. In some embodiments, the light source 802 can indicate a current position of the tip of the needle based on which array of the multi-array ultrasound scanner detects the needle tip crossing its imaging plane. For instance, the multi-array ultrasound scanner can include three arrays: a left array, a center array, and a right array. When the needle crosses the imaging plane of the left array, the light source 802 can emit a green light. However, if the needle tip passes out of the imaging plane of the left array and crosses into the imaging plane of the center array, the light source 802 can change the color of the light emitted to red. In some embodiments, the color of the light emitted by the light source 802 corresponds to the array whose imaging plane the needle tip most recently crossed. For instance, green can indicate that the needle tip has crossed the imaging plane of the center array, red can indicate that the needle tip has crossed the imaging plane of the left array, and orange can indicate that the needle tip has crossed the imaging plane of the right array. Additionally or alternatively, the light source 802 can change its intensity and/or blinking pattern to indicate which array's imaging plane the needle tip has most recently crossed.

In some embodiments, the light source 802 includes multiple light sources spatially separated on the ultrasound scanner, such as side-by-side (not shown in FIG. 8 for clarity). Each of the arrays can correspond to one of the light sources. When the needle crosses the imaging plane of one of the arrays, the system can cause the light source that corresponds to that array to project light. Hence, the user can track the trajectory of the out-of-plane needle insertion by inspection of which light source is projecting light, and/or the color of the light, and/or the blinking pattern of the light.

### Example Transducer Arrays

In some embodiments, an ultrasound scanner, such as a wearable ultrasound scanner or a handheld ultrasound scanner, for determining port health with ultrasound includes a multi-array scanner (e.g., a multi-array transducer). Some embodiments of a multi-array scanner include one or more of the arrays described in U.S. patent application Ser. No. 18/613,694, filed on Mar. 22, 2024, and entitled Multi-Dimensional and Multi-Frequency Ultrasound Transducers to Zhang et al., the disclosure of which is incorporated herein by reference in its entirety. In some embodiments, a multi-array scanner includes one or more of the arrays described in U.S. patent application Ser. No. 17/561,313, filed on Dec. 23, 2021, entitled Array Architecture and Interconnection for Transducers to Li et al., the disclosure of which is incorporated herein by reference in its entirety.

In some embodiments, a multi-array scanner for determining port health with ultrasound includes a first array with array elements selected from the group consisting of PZT, PMUT, and CMUT array elements, and a second array with additional array elements selected from the group consisting of PZT, PMUT, and CMUT array elements. The elements of the first array can be of a different type than the elements of the second array (e.g., the first array can include PMUT or PZT elements, and the second array can include CMUT elements).

In conventional PMUT and CMUT transducers, the vibration mode and operation frequency rely on the structure of the membrane size and thickness. Conventional CMUT transducers generally have a broader bandwidth with more uniform cell size than conventional PMUT arrays. However, the transmitting sensitivity (in terms of power efficiency) of conventional CMUT arrays is usually weaker than conventional PMUT arrays. On the other hand, conventional PMUT arrays usually have better transmitting sensitivity, but narrower bandwidth, than conventional CMUT arrays. To increase the bandwidth, some conventional PMUT arrays use different vibration cell sizes, e.g., different cells contribute acoustic energy with different operation frequencies to reach an overall broader bandwidth. However, this method reduces transmitting sensitivity, and can require significant tuning effort. Thus, conventional PMUT and CMUT arrays usually compromise performance between sensitivity and bandwidth, and may not be suitable for some applications, such as determining port health with ultrasound.

In contrast, systems, devices, and methods for determining port health with ultrasound, including multi-array transducers with combinations of PZT, PMUT, and CMUT arrays that can operate at low and high frequencies based not only on the mechanical structures, but also the electrical complex impedance tuning is disclosed. For example, in some embodiments, the multi-array transducers include one or more PMUT arrays and one or more CMUT arrays. In some embodiments, for each low or high frequency array, the system controls the arrays independently, which facilitates unique imaging modes, including super-harmonic imaging.

In some embodiments, the system includes a multi-section, multi-functional, multi-frequency transducer using multiple uniform vibration sections combined to reach high sensitivity and broader bandwidth. In each section, the cells can have the same mechanical structure. However, the cells can be different between each of the sections. For each PMUT, CMUT, or PZT section, the elements in each functional area can be tuned with the same inductors (e.g., tuning impedances). However, the tuning inductors between each section can be different depending on the desired performance of the overall transducers. The transducer's overall broader bandwidth compared to conventional transducers can be reached through multiple narrow band array sections with each section having a different operation frequency.

The PMUT, CMUT, and/or PZT sections can be tuned differently to enhance the performances. In each section, the cells can be uniformly constructed to provide optimized vibration, and therefore high sensitivity. The overall transducer bandwidth can be reached from combining several sections in the elevational direction, where each section can have a different operation frequency. Each section of the PMUT, CMUT, and/or PZT arrays can be electrically tuned differently to enhance both sensitivity and bandwidth.

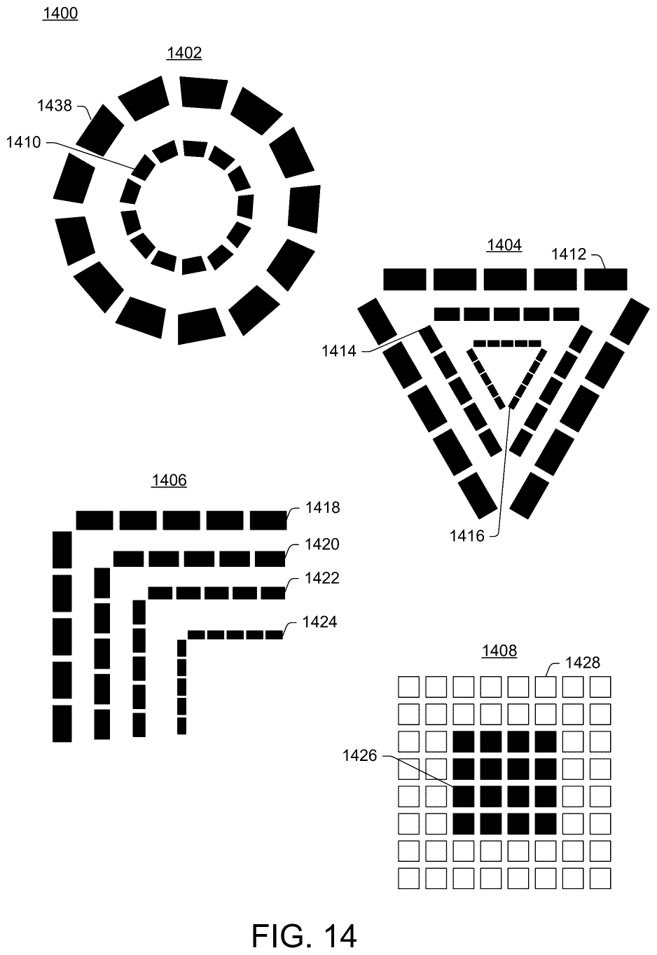

FIG. 9 illustrates some embodiments of a multi-array transducer 900 for determining port health with ultrasound. The multi-array transducer 900 includes three arrays, or sub-arrays, 902, 904, and 906. The first array 902 can be referred to as a center array, as it is between the second array 904 and the third array 906, which can be referred to as adjacent arrays. In this example multi-array transducer 900, the arrays 902-906 are laid out in rows, parallel to one another. As will be discussed below with respect to FIG. 14, multi-array transducers in accordance with the present invention are not so limited. The example multi-array transducer 900 also includes a lens 908 that covers the three arrays 902-906. In the example in FIG. 9, the lens 908 includes multiple radii of curvature. For example, a first radius covers the array 902, a second radius covers the array 904, and a third radius covers the array 906. In an example, the second radius and the third radius are the same radius, which is different than the first radius.

In the example in FIG. 9, the arrays 902-906 of the multi-array transducer 900 include PZT array elements. However, multi-array transducers in accordance with the present invention are not so limited and can include arrays in any suitable combination of PZT, PMUT, and CMUT array elements. In some embodiments, the array 902 can include PZT array elements, and the adjacent arrays 904 and 906 can include PMUT array elements. In another example, the array 902 can include PZT array elements, and the adjacent arrays 904 and 906 can include CMUT array elements. In some other embodiments, the array 902 can include PMUT array elements, and the adjacent arrays 904 and 906 can include CMUT array elements. In another example, the array 902 can include PMUT array elements, and the adjacent arrays 904 and 906 can include PZT array elements.

In some embodiments, the first array 902 operates at a first frequency, and the second and third arrays 904 and 906 operate at a second frequency that is different than the first frequency. For instance, the second frequency can be lower or higher than the first frequency. In some embodiments, the second and third arrays 904 and 906 operate at different frequencies from one another, which can be higher or lower than the first frequency. The frequencies of the arrays 902-906 can be selected so that the bandwidths of the arrays overlap, and so that the union of the individual bandwidths extends the overall bandwidth of the multi-array transducer 900.

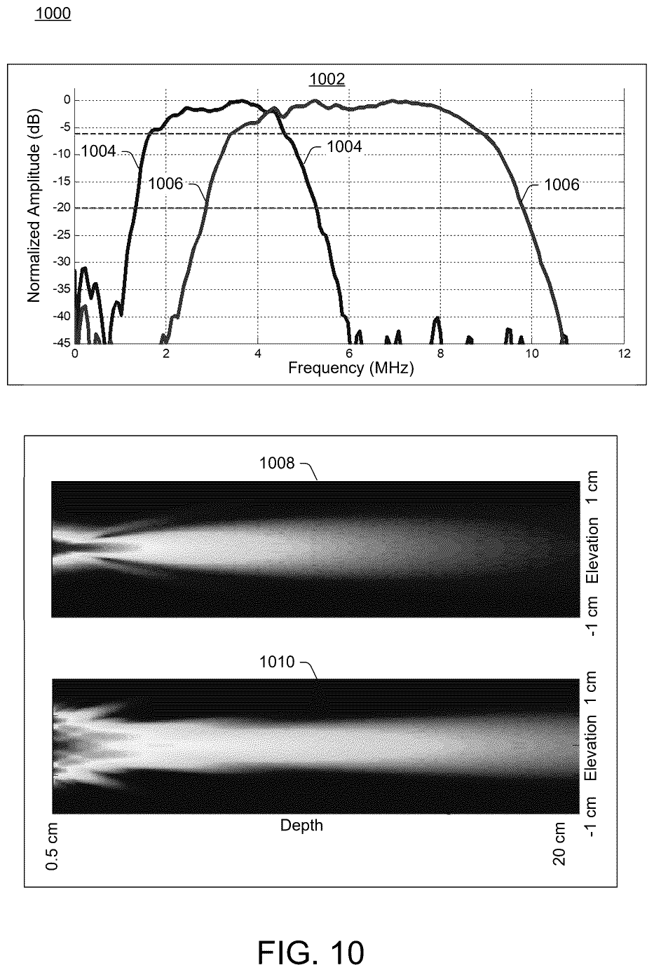

For example, FIG. 10 illustrates example characteristics 1000 of a multi-array transducer for determining port health with ultrasound. The characteristics 1000 include a frequency response 1002 of a multi-array transducer, such as the multi-array transducer 900 in FIG. 9. The frequency response 1002 includes a first bandwidth 1004 and a second bandwidth 1006. The first bandwidth 1004 illustrates the frequency response of an array, such as the arrays 904 and 906 in FIG. 9, and the second bandwidth 1006 illustrates the frequency response of another array, such as the array 902 in FIG. 9. By combining the first bandwidth 1004 and the second bandwidth 1006, the overall bandwidth of the multi-array transducer is increased.

The characteristics 1000 also include illustrations of a first ultrasound beam 1008 and a second ultrasound beam 1010 showing depth against elevation. The first ultrasound beam 1008 corresponds to the array 902 in FIG. 9, and the second ultrasound beam 1010 corresponds to the arrays 904 and 906 in FIG. 9. Because the array 902 is implemented to operate at a higher frequency than the arrays 904 and 906, the second ultrasound beam 1010 has deeper penetration than the first ultrasound beam 1008, but the first ultrasound beam 1008 has better focus than the second ultrasound beam 1010. Hence, the multi-array transducer can exploit the different ultrasound beam profiles associated with the multiple arrays to image at multiple depths with a same ultrasound scanner, rather than requiring the use of multiple ultrasound scanners.

Further, because the transducer includes multiple arrays, these arrays can be implemented to configure the transducer in one of multiple operation (e.g., imaging) modes, as is discussed below with respect to Table 1. Moreover, because the transducer can include multiple arrays of different types of array elements (e.g., PZT, PMUT, and CMUT), the strengths of each of the types of array elements can be exploited. For example, PMUT, which conventionally has better transmit sensitivity than CMUT, can be used for ultrasound transmission, while CMUT, which conventionally has better receive sensitivity than PMUT, can be used for ultrasound reception.

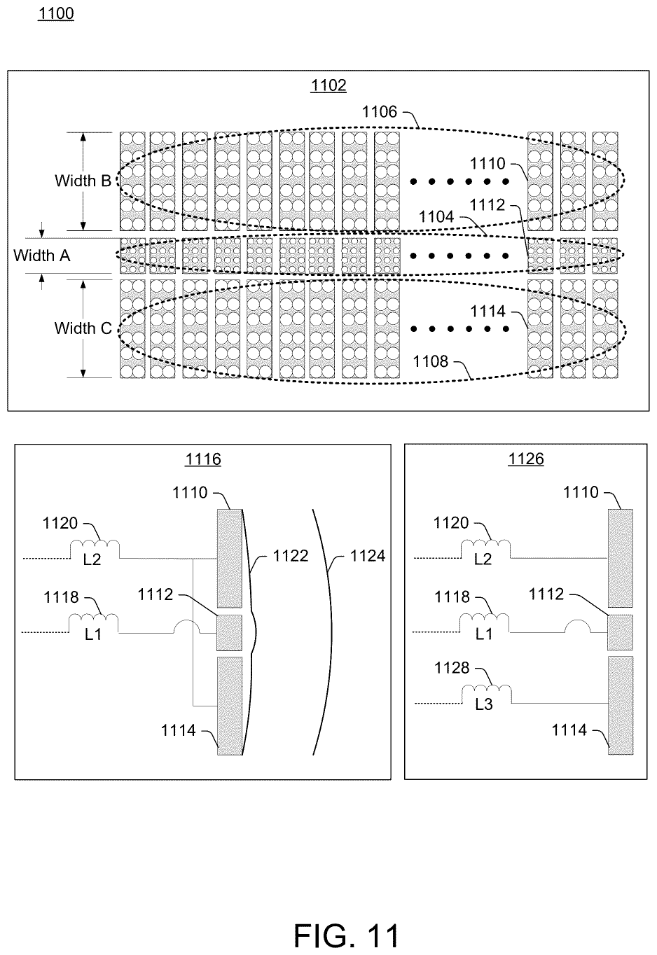

FIG. 11 illustrates some other embodiments of a multi-array transducer 1100 for determining port health with ultrasound. The multi-array transducer 1100 is an example of the multi-array transducer 1000. At inset 1102, the multi-array transducer 1100 includes a first array 1104 (e.g., a center array), and second and third arrays 1106 and 1108 (e.g., adjacent arrays). Each array includes multiple array elements, or sections. For example, section 1110 is an array element of the array 1106, section 1112 is an array element of the array 1104, and section 1114 is an array element of the array 1108. The sections of an array can include any type of array element. For instance, the arrays 1106 and 1108 can include CMUT array elements, and the array 1102 can include PMUT array elements. In another example, the arrays 1102-1106 can each be comprised of PMUT, CMUT, or PZT array elements. The array elements can be comprised of cells, which in this example are illustrated as circles for clarity. However, the cells can be of any suitable shape, such as ellipses, squares, rectangles, polygons, etc.

In the example multi-array transducer 1100, the array 1104 has a width of A, the array 1106 has a width of B, and the array 1108 has a width of C. In some embodiments, including when the arrays 1104 and 1106 (e.g., the adjacent arrays) are implemented to operate at a same frequency, the width B and the width C can be the same width. In some other embodiments, including when the arrays 1104 and 1106 are implemented to operate at different frequencies than one another, the width B can be different from the width C.

The array elements can be tuned to achieve a bandwidth for the array, and the tuning can include to couple a complex impedance to the array element. In some embodiments, each array element (or section) of an array is tuned with a same complex impedance. For instance, inset 1116 illustrates the sections 1110-1114 from the arrays 1104-1108, respectively. For clarity, the cells (circles) are omitted. Each of the sections 1110-1114 at inset 1116 are coupled to a complex impedance. For example, a complex impedance 1118 is coupled to the section 1112, and the complex impedance 1120 is coupled to both the sections 1110 and 1114. The sections 1110 and 1114 are coupled to the same complex impedance 1120 because in this example, width B and width C are equal, and the arrays 1106 and 1108 (e.g., the adjacent arrays) are implemented to operate at a same frequency as one another.

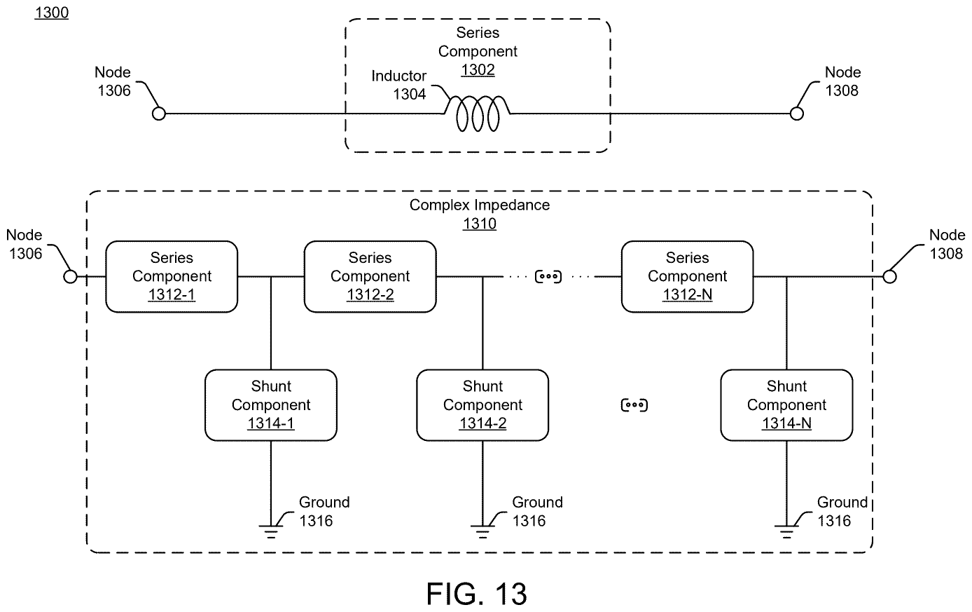

The complex impedances 1118 and 1120 in this example are illustrated for clarity as single inductors with values L1 and L2, respectively. However, as will become apparent below with regards to the discussion of FIG. 13, the complex impedances 1118 and 1120 are not limited to a single element or to just inductors, but can instead include any suitable combination and number of inductors, capacitors, and resistors in series and shunt configurations.

Also at inset 1116, the example multi-array transducer 1100 includes a lens 1122. The lens 1122 includes multiple radii of curvature and is an example of the lens 908. Additionally or alternatively, the multi-array transducer 1100 includes the lens 1124, which includes a single radius of curvature. A lens (e.g., the lens 1122 or the lens 1124) can cover the arrays 1104-1108.

In contrast to inset 1116 which illustrates an implementation of complex tuning impedances for the case when width B and width C are equal, the inset 1126 illustrates an implementation of complex tuning impedances for the case when width B and width C are not equal. In this case, the arrays 1106 and 1108 (e.g., the adjacent arrays) can be implemented to operate at different frequencies from one another. Accordingly, at inset 1126 each of the sections 1110, 1112, and 1114 are coupled to different complex impedances 1120, 1118, and 1128, respectively.

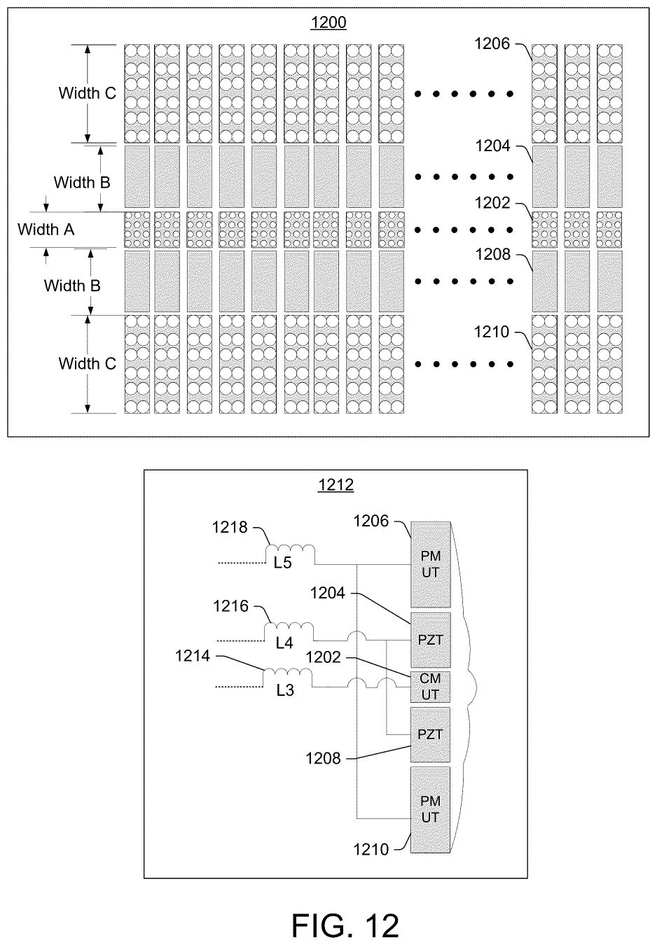

FIG. 12 illustrates some other embodiments of a multi-array transducer 1200 for determining port health with ultrasound. Referring to FIG. 12, the multi-array transducer 1200 is similar to the multi-array transducer 1100, but includes five arrays instead of three. For instance, the array element (or section) 1202 belongs to a center array having a width A. The array element (or section) 1204 belongs to an upper adjacent array having a width B. The array element (or section) 1206 belongs to an upper outer array having a width C. On the lower side of the center array, the array element (or section) 1208 belongs to a lower adjacent array having a width B, and the array element (or section) 1210 belongs to a lower outer array having a width C. In some embodiments, the center array of width A is configured to operate at a first frequency, the adjacent arrays of width B are configured to operate at a second frequency, and the outer arrays of width C are configured to operate at a third frequency. The second and third frequencies can be the same or different from one another and be lower or higher than the first frequency.

Inset 1212 illustrates an implementation of complex tuning impedances for the multi-array transducer 1200. The array elements of the center array, including the array element 1202, are coupled to a complex impedance 1214 (e.g., an inductor with value L3). The array elements of the adjacent arrays, including the array elements 1204 and 1208, are coupled to a complex impedance 1216 (e.g., an inductor with value L4). The array elements of the outer arrays, including the array elements 1206 and 1210, are coupled to a complex impedance 1218 (e.g., an inductor with value L5). As discussed below with respect to FIG. 13, the complex impedances 1214-1218 are not limited to a single element or to just inductors, but can instead include any suitable combination and number of inductors, capacitors, and resistors in series and shunt configurations.

In the example illustrated at the inset 1212, the multi-array transducer 1200 includes arrays comprised of PZT, PMUT, and CMUT array elements. For instance, the array elements of the center array, including the array element 1202, include CMUT array elements. The array elements of the adjacent arrays, including the array elements 1204 and 1208, include PZT array elements. The array elements of the outer arrays, including the array elements 1206 and 1210, include PMUT array elements. However, some embodiments of multi-array transducers for use in determining port health include any combination of arrays of PZT, PMUT, and/or CMUT array elements. For instance, the center array can include CMUT array elements, and both the adjacent arrays and the outer arrays can include PMUT array elements. In another example, the center array can include PZT array elements, upper arrays can include CMUT array elements, and lower arrays can include PMUT array elements.

FIG. 13 illustrates some embodiments of tuning impedances 1300 for transducer arrays for determining port health with ultrasound. The tuning impedances 1300 include a series component 1302 that represents a complex impedance. In some embodiments, the series component 1302 includes a single inductor 1304 between the nodes 1306 and 1308. The inductor 1304 is an example of the inductors 1118, 1120, 1128, and 1214-1218.

The inductor 1304 is illustrated as an example circuit element, and generally the series component 1302 can include any suitable circuit element, such as inductor, capacitor, resistor, combinations thereof, and the like. Further, the series component 1302 can instead include any suitable combination and number of inductors, capacitors, resistors, or other series components in series and shunt configurations, as is illustrated by the complex impedance 1310.

In some embodiments, the complex impedance 1310 includes a series of series components 1312-1, 1312-N between the nodes 1306 and 1308. In some embodiments, between each of the series components 1312-1, 1312-N, the complex impedance 1310 includes one end of one of the shunt components 1314-1-1314-N. The other ends of the shunt components 1314-1-1314-N are connected to electrical ground 1316. Each of the series components 1312-1, 1312-N and the shunt components 1314-1-1314-N can include any suitable circuit element, such as inductor, capacitor, resistor, combinations thereof, and the like. Accordingly, the complex impedance 1310 can be implemented to achieve any suitable tuning impedance for array elements of a multi-array transducer.

FIG. 14 illustrates some embodiments of an array configurations 1400 for multi-array transducers of ultrasound scanners for determining port health with ultrasound. The discussions of arrays of a multi-array scanner above largely focus on arrays comprised of rows of array elements, as illustrated in FIGS. 9, 11, and 12. However, the techniques disclosed herein are not limited to arrays (or sub-arrays) arranged in rows as previously described, but can also include multiple arrays in various configurations. For example, the example array configurations 1400 include multi-dimensional array architectures in accordance with some embodiments. The array configurations 1400 include a circular array configuration 1402, a polygonal array configuration 1404, an open-shaped array configuration 1406, and a matrix array configuration 1408. The arrays in the array configurations 1400 can include any suitable combination of PZT, PMUT, and CMUT array elements.

In some embodiments, the circular array configuration 1402 includes an outer array 1438 of transducer elements and an inner array 1410 of transducer elements arranged in concentric circles. Although circles are illustrated in the circular array configuration 1402, the outer array 1438 and the inner array 1410 can include elements arranged in concentric ellipses in some embodiments. Further, the circular array configuration 1402 can include more than the two concentric arrays that are illustrated. In one example, the inner array 1410 includes CMUT array elements, and the outer array 1438 includes PMUT array elements. In another example, the inner array 1410 includes PMUT array elements, and the outer array 1438 includes CMUT array elements.

In some embodiments, the polygonal array configuration 1404 includes three nested arrays of triangular shape, including an outer array 1412 of transducer elements, a center array 1414 of transducer elements, and an inner array 1416 of transducer elements. The triangular shapes of the three arrays of the polygonal array configuration 1404 are examples of polygons and are meant to be exemplary. Other polygonal shapes that can be included in the polygonal array 1404 include nested arrays arranged in rectangular, rhombus, pentagon, and the like shapes. In some embodiments, the center array 1414 includes CMUT array elements, and the outer array 1412 and the inner array 1416 include PMUT array elements. In another example, the center array 1414 includes PMUT array elements, and the outer array 1412 and the inner array 1416 include CMUT array elements.

In some embodiments, the open-shaped array configuration 1406 includes four nested arrays of L-shapes, including a first outer array 1418 of transducer elements, a second outer array 1420 of transducer elements, a first inner array 1422 of transducer elements, and a second inner array 1424 of transducer elements. The L-shapes of the four arrays of the open-shaped array 1406 are examples of open shapes and are meant to be exemplary. Other open shapes that can be included in the open-shaped array 1406 include nested arrays arranged in C-shapes, V-shapes, S-shapes, and the like. The first outer array 1418, second outer array 1420, first inner array 1422, and second inner array 1424 can include any suitable combination of PMUT, CMUT, and PZT array elements.

In some embodiments, the matrix array configuration 1408 includes an inner array 1426 having array elements on a grid, and an outer array 1428 having array elements on the grid and that surround the array elements of the inner array 1426. In an example, the inner array 1426 is centrally located within the outer array 1428. The inner array 1426 can include PMUT array elements that operate at a first frequency, and the outer array 1428 can include CMUT array elements that operate at a second frequency. In another example, the inner array 1426 can include CMUT array elements that operate at a third frequency, and the outer array 1428 can include PMUT array elements that operate at a fourth frequency. In some embodiments, the inner array 1426 operates at a higher frequency than the outer array 1428. In some embodiments, the inner array 1426 operates at a higher frequency than any other arrays of the matrix array configuration 1408.

In some embodiments, the matrix array configuration 1408 includes a third array (not shown for clarity) that surrounds the outer array 1428. The outer array 1428 can be centered within the third array. In another example, the matrix array configuration 1408 includes a fourth array (not shown for clarity) that surrounds the third array, and the third array can be centered within the fourth array. Hence, the matrix array configuration 1408 can include any suitable number of nested arrays. In an example, the matrix array configuration 1408 includes at least three arrays, including at least one PZT array, at least one PMUT array, and at least one CMUT array.

Table 1 illustrates operation (e.g., imaging) modes and transducer array configurations for a three-array transducer array, e.g., as is illustrated in FIG. 11. The center array represents the transducer array 1104, the first adjacent array represents the transducer array 1106, and the second adjacent array represents the transducer array 1108.

TABLE 1
Example operation (e.g., imaging) modes and transducer
array configurations for a three-array transducer array

| | Near Field | | | Far Field | | |
|---|---|---|---|---|---|---|
| | First | | Second | First | | Second |
| Operation | Adjacent | Center | Adjacent | Adjacent | Center | Adjacent |
| Mode | Array | Array | Array | Array | Array | Array |
| Mode 1 | Not Used | High Freq. | Not Used | Low Freq. | Not | Low Freq. |
| (Linear and | | (Tx/Rx) | | (Tx/Rx) | Used | (Tx/Rx) |
| Phased) | | (Linear) | | (Phased) | | (Phased) |
| Mode 2 | Low Freq. | Linear | Low Freq. | Phased | Linear | Phased |
| (Broadband | (Tx) | High Freq. | (Tx) | Low Freq. | High Freq. | Low Freq. |
| THI) | | (Tx/Rx) | | (Tx) | (Rx) | (Tx) |
| Mode 3 | Low Freq. | Linear | Low Freq. | Phased | Linear | Phased |
| (Full | (Tx/Rx) | High Freq. | (Tx/Rx) | Low Freq. | High Freq. | Low Freq. |
| Aperture | | (Tx/Rx) | | (Tx/Rx) | (Tx/Rx) | (Tx/Rx) |
| and | | | | | | |
| Broadband | | | | | | |
| THI) | | | | | | |

### Example Machine-Learned Model

Many of the aspects described herein can be implemented using a machine-learned model. For the purposes of this disclosure, a machine-learned model is any model that accepts an input, analyzes and/or processes the input based on an algorithm derived via machine-learning training, and provides an output. A machine-learned model can be conceptualized as a mathematical function of the following form:

$$f ( \hat{s} , θ ) = \hat{y} \qquad Equation ( 1 )$$

In Equation (1), the operator f represents the processing of the machine-learned model based on an input and providing an output. The term ŝ represents a model input, such as ultrasound data. The model analyzes/processes the input s using parameters θ to generate output ŷ (e.g., object identification, object segmentation, object classification, etc.). Both ŝ and ŷ can be scalar values, matrices, vectors, or mathematical representations of phenomena such as categories, classifications, image characteristics, the images themselves, text, labels, or the like. The parameters θ can be any suitable mathematical operations, including but not limited to applications of weights and biases, filter coefficients, summations or other aggregations of data inputs, distribution parameters such as mean and variance in a Gaussian distribution, linear algebra-based operators, or other parameters, including combinations of different parameters, suitable to map data to a desired output.

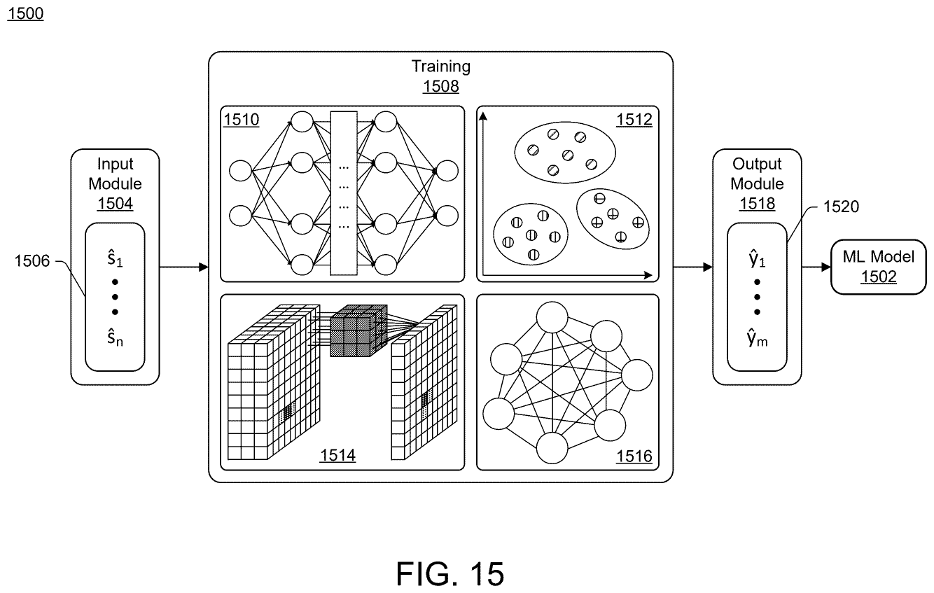

FIG. 15 represents some embodiments of a machine-learning architecture 1500 used to train a machine-learned model M 1502. An input module 1504 accepts an input ŝ 1506, which can be an array with members ŝ₁ through ŝₙ. The input ŝ 1506 is fed into a training module 1508, which processes the input ŝ 1506 based on the machine-learning architecture 1500. For example, if the machine-learning architecture 1500 uses a multilayer perceptron (MLP) model 1510, the training module 1508 applies weights and biases to the input ŝ 1506 through one or more layers of perceptrons, each perceptron performing a fit using its own weights and biases according to its given functional form. MLP weights and biases can be adjusted so that they are optimized against a least mean square, logcosh, or other optimization function (e.g., loss function) known in the art. Although an MLP model 1510 is described here as an example, any suitable machine-learning technique can be employed, some examples of which include but are not limited to k-means clustering 1512, convolutional neural networks (CNN) 1514, a Boltzmann machine 1516, Gaussian mixture models (GMM), and long short-term memory (LSTM). The training module 1508 provides an input to an output module 1518. The output module 1518 analyzes the input from the training module 1508 and provides an output in the form of ŷ 1520, which can be an array with members ŷ₁ through ŷₘ. The output ŷ 1520 can represent a known correlation with the input ŝ 1506, such as, for example, object identification, segmentation, and/or classification.

In some examples, the input ŝ 1506 can be a training input labeled with known output correlation values, and these known values can be used to optimize the output ŷ 1520 in training against the optimization/loss function. In other examples, the machine-learning architecture 1500 can categorize the output ŷ 1520 values without being given known correlation values to the inputs ŝ 1506. In some examples, the machine-learning architecture 1500 can be a combination of machine-learning architectures. By way of example, a first network can use the input ŝ 1506 and provide the output ŷ 1520 as an input ŝ_{ML} to a second machine-learned architecture, with the second machine-learned architecture providing a final output ŷ_{f}. In another example, one or more machine-learning architectures can be implemented at various points throughout the training module 1508.

In some machine-learned models, all layers of the model are fully connected. For example, all perceptrons in an MLP model act on every member of ŝ. For an MLP model with a 100×100 pixel image as the input, each perceptron provides weights/biases for 10,000 inputs. With a large, densely layered model, this may result in slower processing and/or issues with vanishing and/or exploding gradients. A CNN, which may not be a fully connected model, can process the same image using 5×5 tiled regions, requiring only 25 perceptrons with shared weights, giving much greater efficiency than the fully connected MLP model.

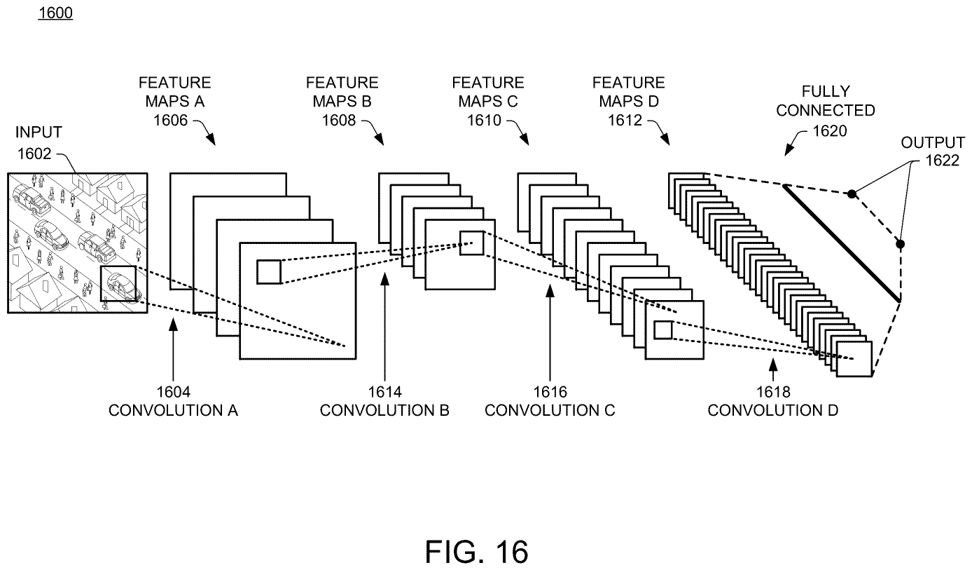

FIG. 16 represents some embodiments of a model 1600 using a CNN to process an input image 1602, which includes representations of objects that can be identified via object recognition, such as people or cars (or an anatomy, as described in relation to FIGS. 1-15). Convolution A 1604 can be performed to create a first set of feature maps (e.g., feature maps A 1606). A feature map can be a mapping of aspects of the input image 1602 given by a filter element of the CNN. This process can be repeated using feature maps A 1606 to generate further feature maps B 1608, feature maps C 1610, and feature maps D 1612 using convolution B 1614, convolution C 1616, and convolution D 1618, respectively. In this example, the feature maps D 1612 become an input for fully connected network layers 1620. In this way, the machine-learned model can be trained to recognize certain elements of the image, such as people, cars, or a particular patient anatomy, and provide an output 1622 that, for example, identifies the recognized elements. In some embodiments, an inference generated with an ultrasound system can be appended to a feature map (e.g., feature map B 1608) generated by a neural network (e.g., CNN). In this way, the feature vector and/or inference can be used as a secondary/conditional input to the neural network.

Although the example of FIG. 16 shows a CNN as a part of a fully connected network, other architectures are possible and this example should not be seen as limiting. There can be more or fewer layers in the CNN. A CNN component for a model can be placed in a different order, or the model can contain additional components or models. There may be no fully connected components, such as a fully convolutional network. Additional aspects of the CNN, such as pooling, downsampling, upsampling, or other aspects known to people skilled in the art can also be employed.

### Example Devices

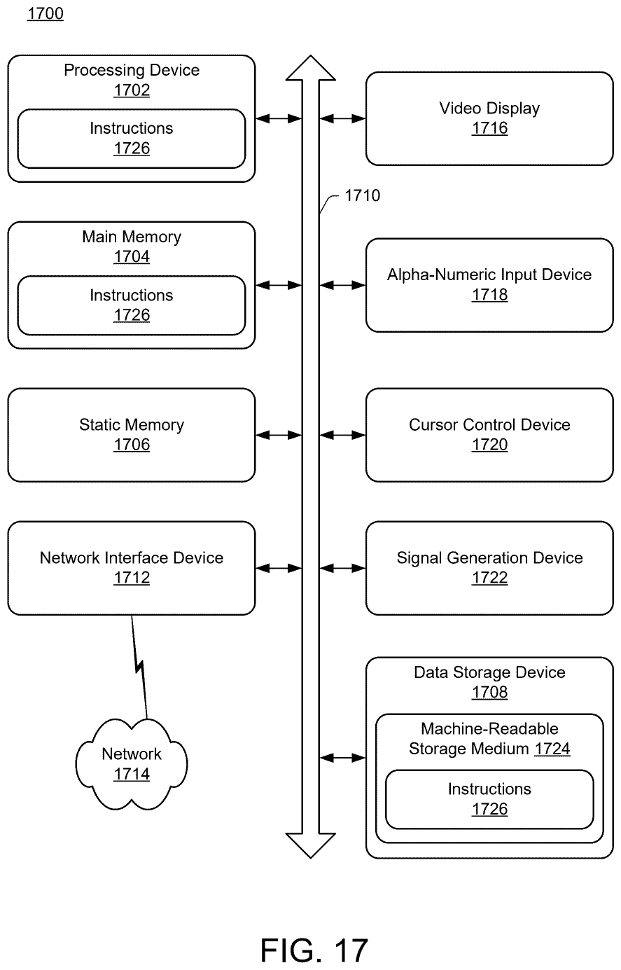

FIG. 17 illustrates a block diagram of some embodiments of a computing device 1700 that can perform one or more of the operations described herein, in accordance with some implementations. The computing device 1700 can be connected to other computing devices in a local area network (LAN), an intranet, an extranet, and/or the Internet. The computing device can operate in the capacity of a server machine in a client-server network environment or in the capacity of a client in a peer-to-peer network environment. The computing device can be provided by a personal computer (PC), a server computer, a desktop computer, a laptop computer, a tablet computer, a smartphone, an ultrasound machine, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein. In some implementations, the computing device 1700 is one or more of an ultrasound machine, an ultrasound scanner, an access point, a charging station, and a medical archiver.

The example computing device 1700 can include a processing device 1702 (e.g., a general-purpose processor, a programmable logic device (PLD), etc.), a main memory 1704 (e.g., synchronous dynamic random-access memory (DRAM), read-only memory (ROM), etc.), and a static memory 1706 (e.g., flash memory, a data storage device 1708, etc.), which can communicate with each other via a bus 1710. The processing device 1702 can be provided by one or more general-purpose processing devices such as a microprocessor, a central processing unit, or the like. In an illustrative example, the processing device 1702 comprises a complex instruction set computing (CISC) microprocessor, a reduced instruction set computing (RISC) microprocessor, a very long instruction word (VLIW) microprocessor, or a processor implementing other instruction sets or processors implementing a combination of instruction sets. The processing device 1702 can also comprise one or more special-purpose processing devices such as an application-specific integrated circuit (ASIC), a field programmable gate array (FPGA), a digital signal processor (DSP), a network processor, or the like. The processing device 1702 can be configured to execute the operations described herein, in accordance with one or more aspects of the present disclosure, for performing the operations and steps discussed herein.

The computing device 1700 can further include a network interface device 1712, which can communicate with a network 1714. The computing device 1700 also can include a video display unit 1716 (e.g., a liquid crystal display (LCD), an organic light-emitting diode (OLED), a cathode ray tube (CRT), etc.), an alphanumeric input device 1718 (e.g., a keyboard), a cursor control device 1720 (e.g., a mouse), and an acoustic signal generation device 1722 (e.g., a speaker, a microphone, etc.). In one embodiment, the video display unit 1716, the alphanumeric input device 1718, and the cursor control device 1720 can be combined into a single component or device (e.g., an LCD touch screen).

The data storage device 1708 can include a computer-readable storage medium 1724 on which can be stored one or more sets of instructions 1726 (e.g., instructions for carrying out the operations described herein, in accordance with one or more aspects of the present disclosure). The instructions 1726 can also reside, completely or at least partially, within the main memory 1704 and/or within the processing device 1702 during execution thereof by the computing device 1700, where the main memory 1704 and the processing device 1702 also constitute computer-readable media. The instructions can further be transmitted or received over the network 1714 via the network interface device 1712.

Various techniques are described in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. In some aspects, the modules described herein are embodied in the data storage device 1708 of the computing device 1700 as executable instructions or code. Although represented as software implementations, the described modules can be implemented as any form of a control application, software application, signal processing and control module, hardware, or firmware installed on the computing device 1700.

While the computer-readable storage medium 1724 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding, or carrying a set of instructions for execution by the machine and that causes the machine to perform the methods described herein. The term “computer-readable storage medium” shall accordingly be taken to include, but not be limited to, solid-state memories, optical media, and magnetic media.

### Example Procedures

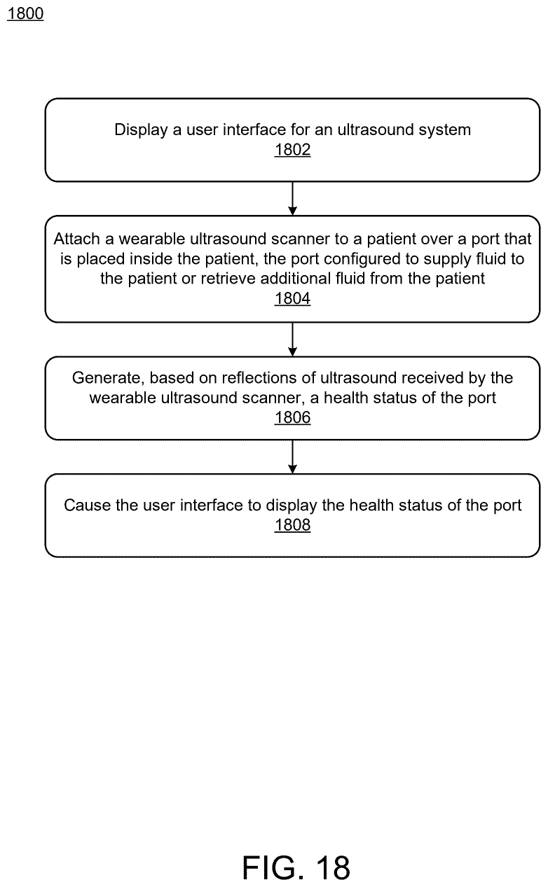

FIG. 18 illustrates some embodiments of a method 1800 that can be implemented by an ultrasound system (e.g., the ultrasound system in the environment 100, the ultrasound system in the implementation 200, and the ultrasound system in the environment 300) for determining port health with ultrasound. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 17.

A user interface for an ultrasound system is displayed (block 1802). For instance, a display device (e.g., the display device 108 and/or the display 412) can display the user interface. A wearable ultrasound scanner is attached to a patient over a port that is placed inside the patient, the port configured to supply fluid to the patient or retrieve additional fluid from the patient (block 1804). Based on reflections of ultrasound received by the wearable ultrasound scanner, a health status of the port is generated (block 1806). The user interface is caused to display the health status of the port (block 1808).

In some embodiments, the processor system generates the health status of the port including a prediction of when the port will fail. The prediction can include a number of days, hours, months, etc. that represents an expected time to failure for the port.

In some embodiments, the system includes a transceiver. The processor system can cause the transmitter to communicate, over a network, the health status to the display device to cause the user interface to display the health status. Additionally or alternatively, the processor system can determine, based on the health status of the port, transmission times. The transmission times can include periodic times, such as every 24 hours, times that are not periodic, such as in one day, three days, and five days. The processor system can generate additional health statuses of the port, and communicate, at the transmission times, the additional health statuses over the network to at least one of the display device and a medical archiver.

In aspects of determining port health with ultrasound, in some embodiments, the processor system generates the health status of the port based on at least one of an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of the fluid or the additional fluid. The processor can implement a machine-learned model to generate the health status of the port. The machine-learned model can include a convolutional neural network, and one or more of the indication of infection of tissue proximate to the port, the amount of swelling of the tissue, the indication of congestion in the port, the measure of volume flow of the fluid or the additional fluid, a measure of the temperature of tissue proximate to the port, and the measure of pressure of the fluid or the additional fluid can be concatenated into an input vector that is processed by the convolutional neural network. Alternatively, some of these parameters can be can be concatenated into an input vector that is processed by layers of the convolutional neural network. The others of these parameters can be provided as conditional inputs to the convolutional neural network and concatenated with a feature vector that the convolutional neural network generates at the output of said layers of the convolutional neural network. The resulting concatenated vector can be processed by additional layers of the convolutional neural network.

In some embodiments, the wearable ultrasound scanner includes a patch that includes the processor system and a coupling agent implemented to couple the ultrasound from the wearable ultrasound scanner to the patient and couple the reflections from the patient to the wearable ultrasound scanner. The wearable ultrasound scanner can include a first transducer array and a second transducer array. The first transducer array can be implemented to transmit the ultrasound and the second transducer array can be implemented to receive the reflections of the ultrasound. In some embodiments, the first transducer array includes at least one of piezoelectric micromachined ultrasonic transducer (PMUT) array elements and lead zirconate titanate (PZT) array elements, and the second transducer array includes capacitive micromachined ultrasonic transducer (CMUT) array elements. The first transducer array can be implemented to operate at a first ultrasound frequency and the second transducer array can be implemented to operate at a second ultrasound frequency that is different from the first ultrasound frequency. In some embodiments, the first transducer array and the second transducer array are removably attached to the patch so that the first transducer array and the second transducer array can be removed and reattached to the patch at different positions on the patch.

In some embodiments, the wearable ultrasound scanner includes a patch and the display device is implemented to be removably attached to the patch. The patch can be disposable and the display device can be sterilized for reuse.

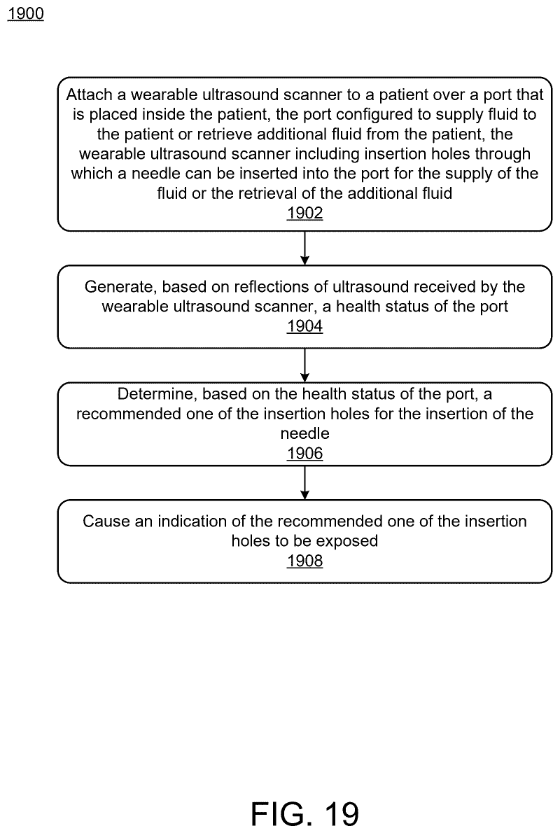

FIG. 19 illustrates some embodiments of a method 1900 that can be implemented by an ultrasound system (e.g., the ultrasound system in the environment 100, the ultrasound system in the implementation 200, and the ultrasound system in the environment 300) for determining port health with ultrasound. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 17.

A wearable ultrasound scanner is attached to a patient over a port that is placed inside the patient, where the port configured to supply fluid to the patient or retrieve additional fluid from the patient, the wearable ultrasound scanner including insertion holes through which a needle can be inserted into the port for the supply of the fluid or the retrieval of the additional fluid (block 1902). Based on reflections of ultrasound received by the wearable ultrasound scanner, a health status of the port is generated (block 1904). Based on the health status of the port, a recommended one of the insertion holes for the insertion of the needle is determined (block 1906). An indication of the recommended one of the insertion holes is caused to be exposed (block 1908).

In some embodiments, the wearable ultrasound scanner includes light sources proximate to the insertion holes. The exposure of the indication of the recommended one of the insertion holes can include to activate a light source of the light sources that is proximate to the recommended one of the insertion holes.

In some embodiments, the system includes a display device implemented to display the indication of the recommended one of the insertion holes. The wearable ultrasound scanner can include the display device. In some embodiments, the display device can display an orientation for the needle for the insertion through the recommended one of the insertion holes.

The processor system can generate the health status of the port based on at least one of an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of the fluid or the additional fluid. For instance, the processor system can implement one or more machine-learned models that process these parameters as an input vector at a top layer of a CNN and/or as an input vector at the top layer and a conditional input vector input at a subsequent layer of the CNN, as previously described.

In some embodiments, the processor system is implemented to track, based on the reflections of the ultrasound, a tip of the needle. The processor system can then indicate, via a light source on the wearable ultrasound scanner, a current position of the tip of the needle. In some embodiments, the wearable ultrasound scanner includes multiple transducer arrays, and the processor system can determine one transducer array of the multiple transducer arrays that covers the current position of the tip of the needle, and select, based on the one transducer array, the light source from among multiple light sources on the wearable ultrasound scanner. In an example, the wearable ultrasound scanner includes multiple transducer arrays, and the processor system is implemented to determine one transducer array of the multiple transducer arrays that covers the current position of the tip of the needle, and select, based on the one transducer array, a color or lighting pattern of the light source.

In some embodiments, the processor system determines, based on the health status of the port, an additional one of the insertion holes not recommended to use for the insertion of the needle. The processor system can cause to be exposed an indication that the additional one of the insertion holes is not recommended for the insertion of the needle. For instance, the processor system can cause the indication to be exposed on a user interface of a display device, such as an ultrasound machine or the wearable ultrasound scanner.

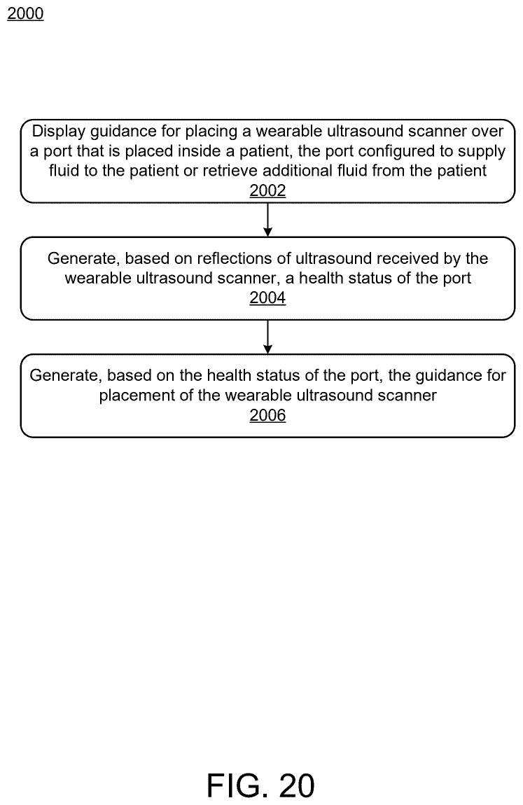

FIG. 20 illustrates some embodiments of a method 2000 that can be implemented by an ultrasound system (e.g., the ultrasound system in the environment 100, the ultrasound system in the implementation 200, and the ultrasound system in the environment 300) for determining port health with ultrasound. The ultrasound system can include an ultrasound scanner (e.g., transducer or probe), an ultrasound machine, a processor system, and a display device. In some embodiments, the ultrasound system includes a computing device having processing logic that can include hardware (e.g., circuitry, dedicated logic, memory, etc.), software (such as is run on a general-purpose computer system or a dedicated machine), firmware (e.g., software programmed into a read-only memory), or combinations thereof. In some embodiments, the process is performed by one or more processors of a computing device such as, for example, but not limited to, an ultrasound machine with an ultrasound imaging subsystem. In some embodiments, the computing device is represented by a computing device as shown in FIG. 17.

Guidance for placing a wearable ultrasound scanner over a port that is placed inside a patient is displayed, the port configured to supply fluid to the patient or retrieve additional fluid from the patient (block 2002). Based on reflections of ultrasound received by a wearable ultrasound scanner, a health status of the port is generated (block 2004). Based on the health status of the port, the guidance for placement of the wearable ultrasound scanner is generated (block 2006).

In some embodiments, the processor system generates the health status of the port based on at least one of an indication of infection of tissue proximate to the port, an amount of swelling of the tissue, an indication of congestion in the port, a measure of volume flow of the fluid or the additional fluid, a measure of the temperature of tissue proximate to the port, and a measure of pressure of the fluid or the additional fluid. For instance, the processor system can implement one or more machine-learned models that process these parameters as an input vector at a top layer of a CNN and/or as an input vector at the top layer and a conditional input vector input at a subsequent layer of the CNN, as previously described.

In some embodiments, the system includes a transceiver implemented to transmit the health status of the port to a nurse station of a care facility. Additionally or alternatively, the transceiver can transmit the health status of the port to a server system maintained by the care facility. Additionally or alternatively, the transceiver can transmit the health status of the port to a medical archiver. Additionally or alternatively, the transceiver can transmit the health status of the port to an ultrasound machine, e.g., a point-of-care ultrasound (POCUS) ultrasound machine. Additionally or alternatively, the transceiver can transmit the health status of the port to a computing device that displays the guidance.

In some embodiments, the fluid includes at least one of a drug, saline fluid, dextrose fluid, lactated Ringer's fluid, and blood. Additionally or alternatively, the additional fluid can include at least one of blood, urine, extracellular fluid, semen, amniotic fluid, cerebrospinal fluid, and plasma.

In aspects of determining port health with ultrasound, in some embodiments, the guidance includes a location and an orientation of the wearable ultrasound scanner, the location and the orientation relative to the port. Additionally or alternatively, the guidance can include an image that depicts a view that should be obtained by the wearable ultrasound device when properly placed. Additionally or alternatively, the wearable ultrasound scanner can include multiple transducer arrays, and the processor system can select, based on the health status of the port, one of the multiple transducer arrays. The processor system can then determine at least one of the location and the orientation so that the one of the multiple transducer arrays is positioned over the port.

In some embodiments, the processor system can, after the wearable ultrasound scanner is placed on the patient based on the guidance, generate, based on additional reflections of ultrasound received by the wearable ultrasound scanner, an additional health status of the port. The additional health status of the port can include an expected time to failure for the port. Additionally or alternatively, the processor system can schedule, based on the expected time to failure, an appointment for the patient for replacement of the port. Additionally or alternatively, the processor system can cause the display device to display a recommendation, based on the expected time to failure, to schedule an appointment for the patient for replacement of the port.

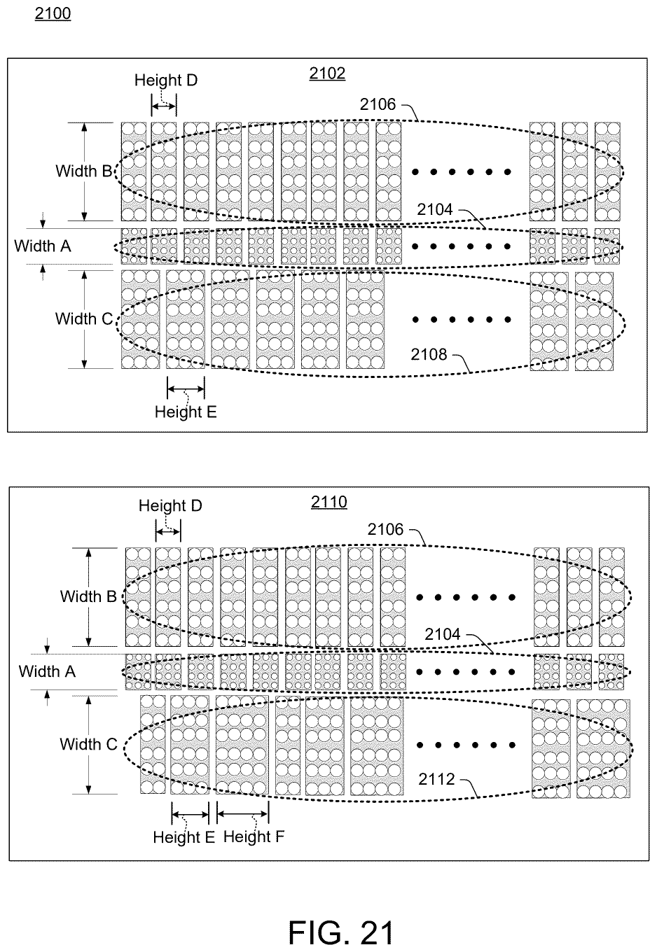

FIG. 21 illustrates example multi-array transducers 2100. The example multi-array transducers 2100 include an example multi-array transducer 2102 that includes a first array 2104 (e.g., a center array), and second and third arrays 2106 and 2108 (e.g., adjacent arrays). In the example multi-array transducer 2102, the array elements of the array 2104 have a width of A, the array elements of the array 2106 have a width of B, and the array elements of the array 2108 have a width of C. The height and width are in the lateral and elevation dimensions across the array, respectively, (looking at the face of the ultrasound scanner from which transmission of ultrasound and reception of reflected ultrasound occurs). In some embodiments, the width B and the width C can be the same width. In other embodiments, the width B can be different from the width C. The widths (and heights) of the array elements can be selected based on the desired frequencies generated by the arrays.

Further, in the example multi-array transducer 2102, the array elements of the array 2104 and the array 2106 have a height of D, and the array elements of the array 2108 have a height of E. Hence, the multi-array transducer 2102 includes arrays arranged in rows that can have array elements of different heights, unlike the multi-array transducer 1100 previously described with respect to FIG. 11. The two heights D and E illustrated in FIG. 21 are meant to be exemplary and non-limiting. For instance, in some embodiments, the multi-array transducer has any suitable number of arrays, e.g., arranged in rows or any suitable shape, and are not limited to the heights illustrated in the example multi-array transducer 2102. As an example, a multi-array transducer can include three arrays, e.g., the arrays 2104-2106, each having array elements of a different height. The array elements can be comprised of cells (e.g., emitters), which in this example are illustrated as circles for clarity. However, the cells can be of any suitable shape, such as ellipses, squares, rectangles, polygons, etc.

Because manufacturing a multi-array transducer having PZT array elements usually involves use of a dicing saw to cut the PZT array elements, it can be difficult to manufacture the multi-array transducer 2102 having PZT elements with different heights. However, because PMUT array elements can be manufactured without the use of a dicing saw, a PMUT array can be manufactured with multiple arrays having array elements of different heights, as is illustrated in the multi-array transducer 2102. The array elements can be tuned with any suitable complex impedance coupled to the array element, as was previously described with respect to FIG. 13.

Further, in some embodiments, the multi-array transducer includes at least one array that has array elements of different heights within the array. For instance, the example multi-array transducers 2100 include an example multi-array transducer 2110 that includes the arrays 2104 and 2106 as described with respect to the multi-array transducer 2102, as well as the array 2112. The array 2112 has array elements with a width of C, like the array 2108. But unlike the array 2108, the array 2112 includes array elements of different heights, including some array elements with a height of E and other array elements with a height of F. By using array elements of different heights, the array 2112 can have a wider bandwidth than an array having elements of all the same height.

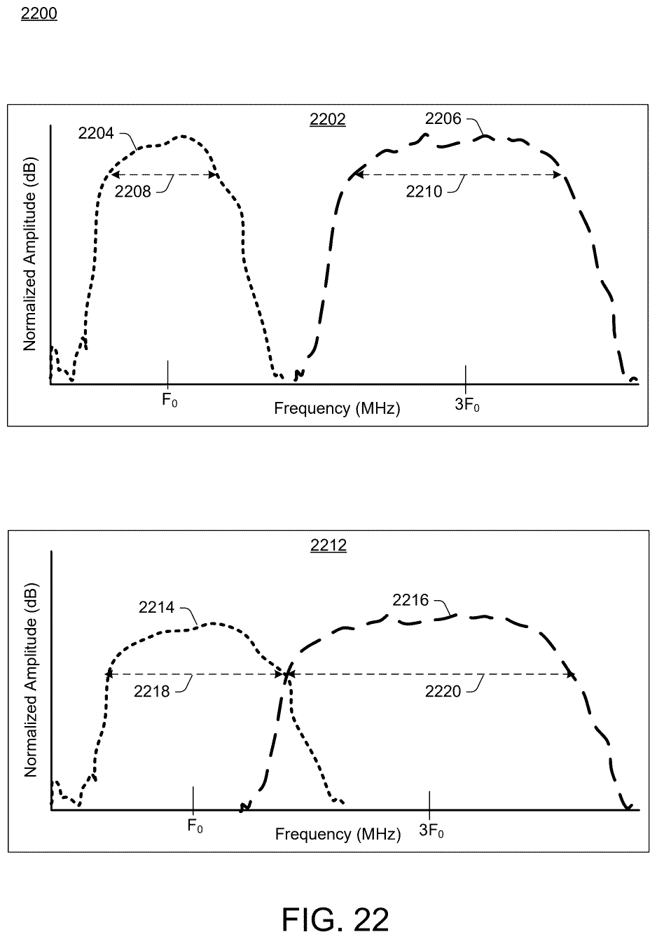

In some embodiments, the array elements of a multi-array transducer have different sized widths and/or heights to set the bandwidths and center frequencies of the arrays of the multi-array transducer. The bandwidths and center frequencies of the arrays can be selected to determine an amount of overlap of the bandwidths of the arrays, e.g., so that they do not overlap, or partially overlap, as is illustrated in FIG. 22, to accommodate different imaging modes, such as harmonic imaging, sub-harmonic imaging, and the like.

FIG. 22 illustrates example characteristics 2200 of some embodiments of a multi-array transducer. The characteristics 2200 illustrated at inset 2202 include a first array response 2204 and a second array response 2206. The array responses 2204, 2206 are examples of frequency responses of arrays of a multi-array transducer. As a non-limiting example, the first array response 2204 can correspond to one of the adjacent arrays illustrated in FIG. 21, such as one of the arrays 2106, 2108, or 2112, and the second array response 2206 can correspond to the center array 2104.

The first array response 2204 has a bandwidth 2208, and the second array response 2206 has a bandwidth 2210. The bandwidths 2208, 2210 can be any suitably defined bandwidth, such as a 3 dB bandwidth, 6 dB bandwidth, etc. Further, the first array response 2204 is centered at a fundamental frequency F₀, and the second array response 2206 is centered at the third harmonic of the fundamental frequency, or 3*F₀. The illustrated center frequencies are meant to be exemplary and non-limiting. The center frequencies and bandwidths 2208, 2210 are such that the first array response 2204 and the second array response 2206 do not overlap.

In some embodiments, the array characteristics illustrated at inset 2202 are suitable for third harmonic imaging. For example, an array with the first array response 2204 can be used for ultrasound transmission, and another array with the second array response 2206 can be used for ultrasound reception, e.g., by receiving a reflection at the third harmonic of the ultrasound transmitted by the array with the first array response 2204. Since the first array response 2204 and the second array response 2206 do not substantially overlap in frequency, noise processes associated with the first array response 2204 are filtered out by the second array response 2206, which can result in better image quality than reception based on the fundamental frequency.

In another example, the array characteristics illustrated at inset 2202 are suitable for sub-harmonic imaging. For instance, an array with the second array response 2206 can be used for ultrasound transmission at the frequency 3*F₀, and an array with the first array response 2204 can be used for ultrasound reception of the sub-harmonic of the frequency 3*F₀, e.g. by receiving at F₀.

Another example of the characteristics 2200 is illustrated at inset 2212, which depicts a third array response 2214 and a fourth array response 2216. The array responses 2214, 2216 are examples of frequency responses of arrays of a multi-array transducer. As a non-limiting example, the third array response 2214 can correspond to one of the adjacent arrays illustrated in FIG. 21, such as one of the arrays 2106, 2108, or 2112, and the fourth array response 2216 can correspond to the center array 2104.

The third array response 2214 has a bandwidth 2218, and the fourth array response 2216 has a bandwidth 2220. The bandwidths 2218, 2220 can be any suitably defined bandwidth, such as a 3 dB bandwidth, 6 dB bandwidth, etc. Further, the third array response 2214 is centered at a fundamental frequency F₀, and the second array response 2216 is centered at the third harmonic of the fundamental frequency, or 3*F₀. The center frequencies and bandwidths 2218, 2220 are such that the third array response 2214 and the fourth array response 2216 partially (or barely) overlap, e.g., they abut against one another so that the upper limit of the bandwidth 2218 matches the lower limit of the bandwidth 2220.

The bandwidths and center frequencies of the array responses 2214, 2216 can be set based on sizes (e.g., heights and widths) of array elements of the arrays corresponding to the array responses 2214, 2216, as previously discussed with respect to FIG. 21. Further, as previously illustrated with respect to the frequency response 1002 in FIG. 10, the sizes of the array elements can be used to set bandwidths and center frequencies of arrays of a multi-array transducer having more substantial overlap. Hence, some embodiments of a multi-array transducer can be manufactured to suit a variety of imaging modes (e.g., harmonic imaging, sub-harmonic imaging, the imaging modes depicted in Table 1, etc.) at different frequencies. In some embodiments, the different frequencies can be suitable for imaging different types of anatomies, such as by using higher frequencies for anatomies at depths closer to the patient's skin, and lower frequencies for anatomies at deeper depths. By including multiple arrays configured for operation at different frequencies and bandwidths in some embodiments of a scanner, a single scanner can be used by a clinician to image different anatomies, unlike conventional ultrasound systems that can require a different ultrasound scanner for different types of anatomies being imaged.

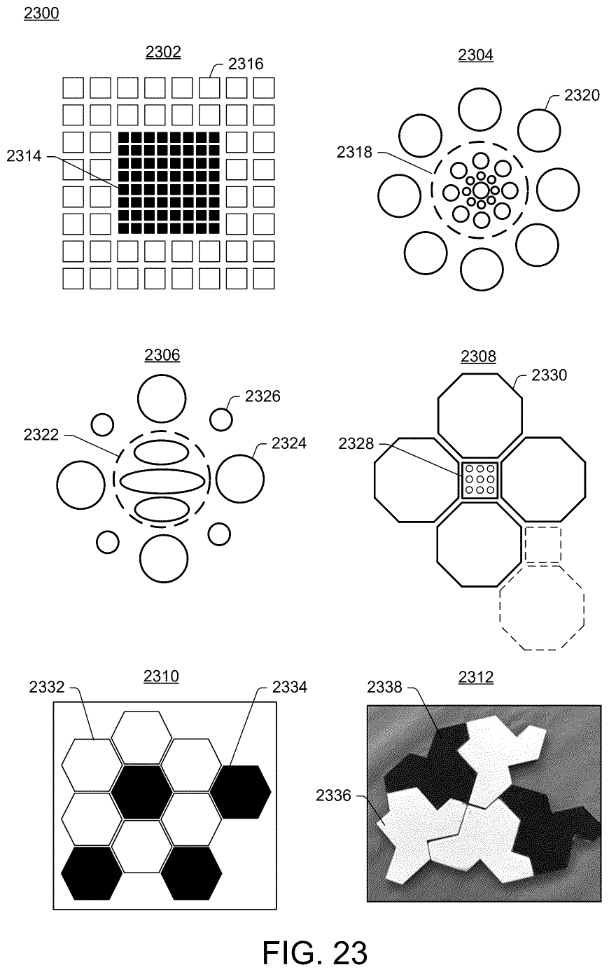

Further, as previously described with respect to FIG. 14, arrays of some embodiments of a multi-array scanner are not limited to multi-array transducers having rows of elements, as is illustrated in FIGS. 9, 11, 12, and 21. Rather, arrays of some embodiments of a multi-array scanner can be of any suitable configuration, with array elements of the same or different size, such as is illustrated in FIG. 23.

FIG. 23 illustrates example array configurations 2300 for some embodiments of a multi-array transducer. The array configurations 2300 include a matrix array configuration 2302, a first circular array configuration 2304, a second circular array configuration 2306, an octagon array configuration 2308, a hexagon array configuration 2310, and an Einstein tile array configuration 2312. The arrays in the array configurations 2300 can include any suitable combination of PZT, PMUT, and CMUT array elements.

The matrix array configuration 2302 includes an inner array 2314 having array elements on a grid, and an outer array 2316 having array elements on the grid and that surround the array elements of the inner array 2314. Unlike the matrix array configuration 1408 illustrated in FIG. 14, the matrix array configuration 2302 in FIG. 23 includes arrays with different size array elements. For instance, the array elements of the inner array 2314 are smaller than the array elements of the outer array 2316. Gaps between the array elements in the arrays can be used for interconnections, through-holes, etc., to connect the array elements to system electronics. Each of the array elements of the matrix array configuration 2302, e.g., each of the squares, can include cells (e.g., emitters), such as the circles illustrated in the array elements of FIG. 21, that are omitted in FIG. 23 for clarity. Further, although the matrix array configuration 2302 includes square array elements, the arrays are not so limited and can include any suitable shape, such as rectangles, ellipses, triangles, polygons, etc.

In an example, the inner array 2314 is centrally located within the outer array 2316. The inner array 2314 can include PMUT array elements that operate at a first frequency, and the outer array 2316 can include CMUT array elements that operate at a second frequency. The first and second frequencies can correspond to a fundamental frequency and a harmonic of the fundamental, such as the third harmonic. In another example, the inner array 2314 can include CMUT array elements that operate at a third frequency, and the outer array 2316 can include PMUT array elements that operate at a fourth frequency. In some embodiments, the inner array 2314 operates at a higher frequency than the outer array 2316. In some embodiments, the inner array 2314 operates at a higher frequency than any other arrays of the matrix array configuration 2302.

In an example, the matrix array configuration 2302 includes a third array (not shown for clarity) that surrounds the outer array 2316. The outer array 2316 can be centered within the third array. In another example, the matrix array configuration 2302 includes a fourth array (not shown for clarity) that surrounds the third array, and the third array can be centered within the fourth array. Hence, the matrix array configuration 2302 can include any suitable number of nested arrays. In an example, the matrix array configuration 2302 includes at least three arrays, including at least one PZT array, at least one PMUT array, and at least one CMUT array.

The first circular array configuration 2304 includes an inner array 2318 and an outer array 2320. The inner array 2318 includes rings of circular array elements of different sizes, and the outer array 2320 includes circular array elements each of a same size that are centered at a same distance from the center of the inner array 2318. The second circular array configuration 2306 includes an inner array 2322 and two outer arrays 2324 and 2326. The inner array 2322 includes elliptical array elements of different sizes. The outer arrays 2324 and 2326 are centered at a same distance from the center of the inner array 2322 and have different size circular array elements from one another that are spaced at the distance so that the larger array elements of the outer array 2324 do not touch the smaller array elements of the outer array 2326. The circular array configurations 2304, 2306 can include any suitable combination of PZT, PMUT, and CMUT arrays. In an example, an inner array includes a PMUT array, and an outer array includes a CMUT array. In another example, the inner and outer arrays include PMUT arrays.

In some embodiments, the circular array configurations 2304, 2306 can be repeated in a plane, and the repeated pattern can be included in an ultrasound scanner. For instance, the inner array 2318 can include multiple copies of the inner array 2318 illustrated in the array configuration 2304, and the outer array 2320 can include multiple copies of the outer array 2320 illustrated in the array configuration 2304. Similarly, the inner array 2322 can include multiple copies of the inner array 2322 illustrated in the array configuration 2306, the outer array 2324 can include multiple copies of the outer array 2324 illustrated in the array configuration 2306, and the outer array 2326 can include multiple copies of the outer array 2326 illustrated in the array configuration 2306.

The octagon array configuration 2308 includes a first array 2330 having octagonal array elements, and a second array 2328 having square array elements. The square array elements of the second array 2328 are located such that each side of a square array element is adjacent (e.g., parallel with) a side of the octagonal array elements. The octagonal array elements of the first array 2330 can be implemented so that the first array 2330 operates at a first frequency and has a first bandwidth, and the square array elements of the second array 2328 can be implemented so that the second array 2328 operates at a second frequency and has a second bandwidth. One of the square array elements of the second array 2328 is illustrated in FIG. 23 with circular cells (e.g., emitters). Another array element of the second array 2328 is illustrated in phantom (dashed lines) in FIG. 23.

The hexagon array configuration 2310 includes a first array 2332 and a second array 2334, each having hexagonal array elements. The hexagonal array elements of the first array 2332 are illustrated as white, and the hexagonal array elements of the second array 2334 are illustrated as black. The array elements of the arrays 2332, 2334 are arranged in a hexagonal closest packing configuration such that the array elements of the first array 2332 abut one another, while the array elements of the second array 2334 do not abut one another, but instead abut array elements of the first array 2332. In an example, the first array 2332 includes array elements of a first type (e.g., PMUT), operates at a first frequency, and has a first bandwidth. The second array 2334 can include array elements of a second type (e.g., PZT), operate at a second frequency, and have a second bandwidth.

The Einstein tile array configuration 2312 includes a first array 2336 and a second array 2338, each having array elements shaped as Einstein tiles. The array elements of the first array 2336 are illustrated as white, and the array elements of the second array 2338 are illustrated as black. The Einstein-tile shaped array elements can cover a plane without repeating a pattern (e.g., there is no kernel pattern of the array elements). Hence, the Einstein tile array configuration 2312 can be less likely to fail, e.g., due to being less likely to crack, and/or propagate a crack, compared to conventional ultrasound arrays that are implemented in regular, repeatable patterns. In an example, the first array 2336 operates at a first frequency and has a first bandwidth, and the second array 2338 operates at a second frequency and has a second bandwidth. The first array 2336 can include array elements of a first type (e.g., PMUT), and the second array 2338 can include array elements of a second type (e.g., CMUT). Additionally or alternatively, the elements of the first array 2336 can be tuned with a first complex impedance, and elements of the second array 2338 can be tuned with a second complex impedance to affect the different frequency responses of the arrays.

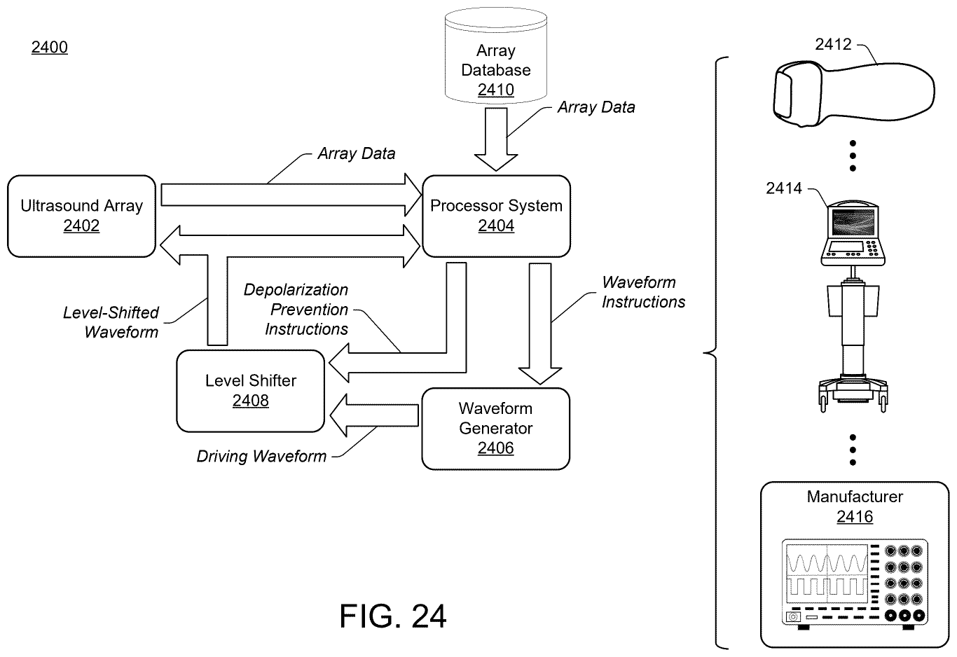

In some embodiments, one or more of the array configurations 2300 are tuned electronically (e.g., by adjusting their driving waveform) so that the array configuration approximates a shape. For example, the voltage of the driving waveform for array elements of the matrix array configuration 2302 can be tuned so that the some of the outer elements are de-emphasized, or even disabled, so that the matrix array configuration 2302 approximates the shape of another array configuration, e.g., the first circular array configuration 2304. In some embodiments, the driving waveforms can be adjusted via the calibration system 2400, discussed below with respect to FIG. 24.

In some embodiments, the resulting shape of an array configuration from the electronic tuning is suitable for harmonic imaging. For instance, by effectively changing the shape of an array configuration, it can generate stronger harmonic content (output) than before the electronic tuning, because certain shapes can result in different side lobes and harmonic content. For instance, an array shape with discontinuities and sharp angles can produce stronger third harmonics than an array shape with continuous, smooth sides. In some embodiments, the array elements of an array configuration are selected so that they have discontinuities and sharp angles, rather than continuous, smooth array elements, so that the third harmonic is enhanced, due to edge effects. For instance, because the Einstein tile array configuration 2312 has array elements with sharp angles, in some embodiments it can generate stronger third harmonics than the first circular array configuration 2304, and therefore be more suitable for harmonic imaging modes.

FIG. 24 illustrates an example calibration system 2400 for some embodiments of a multi-array transducer. The calibration system 2400 can be used to adjust the driving waveform for a multi-array transducer to avoid depolarizing the array when a bi-polar waveform is used. For example, an array, e.g., a PZT array, is polarized by applying a polarization voltage to induce a piezo-electric effect. The polarization voltage is proportional to the thickness of the material of the array. PMUT arrays are generally much thinner than PZT, so require a much lower polarization voltage than PZT, e.g., 5V for PMUT compared to 500V for PZT. If a negative voltage is applied to the array, the piezo-electric effect can be removed, rendering the transducer inoperable. When a bi-polar driving waveform is used, it can accidentally depolarize a PMUT array because the polarization voltage for the PMUT array is small. Accordingly, the calibration system 2400 includes level-shifting depolarization circuitry to avoid accidental depolarization of an array, such as a PMUT array.

Further, the calibration system 2400 can be used for hybrid arrays that include different types of array elements, such as PMUT and PZT elements, to level shift the driving waveform on a per element basis, based on the element type. This can not only avoid accidental depolarization, but can also avoid wasted energy and excess heat, and reduce safety risks. Further, over time an array's performance can degrade for a fixed polarization voltage. For example, sensitivity can decrease over time, and reliability can go down. Hence, to maintain performance over time, the calibration system 2400 can increase the polarization voltage based on the array's age and/or use, (e.g., a younger array can require a lower polarization voltage than an older array for a same performance level).

The calibration system 2400 includes an ultrasound array 2402, which can include one or more ultrasound arrays of a multi-array scanner, such as the scanner 104. In some embodiments, the ultrasound array 2402 includes array elements of different types, such as PMUT and PZT array elements. The calibration system 2400 also includes a processor system 2404, which is an example of the processor(s) 106. The calibration system 2400 also includes a waveform generator 2406, a level shifter 2408, and an array database 2410.

The processor system 2404 receives array data. In some embodiments, the processor system 2404 receives the array data from the ultrasound array 2402. Additionally or alternatively, the processor system 2404 can receive the array data from the array database 2410 that maintains data on ultrasound arrays. The array data can include data that describes the ultrasound array 2402, including an array type (e.g., PZT, PMUT, CMUT, combinations thereof, etc.), an age of the array, a history of use of the array (e.g., a number of hours of operation of the array, a number of cleaning cycles the array has undergone, etc.), calibration data for the array (e.g., a time since a last calibration for the array, a voltage level used in the level shifter 2408 in the last calibration for the array, etc.), and the like.

The processor system 2404 also receives a level-shifted waveform generated by the level shifter 2408. The level shifter 2408 receives depolarization prevention instructions from the processor system 2404, and a driving waveform from the waveform generator 2406 that is generated based on waveform instructions from the processor system 2404. Based on the depolarization prevention instructions from the processor system 2404, the level shifter 2408 level shifts the driving waveform generated by the waveform generator 2406 so that the level-shifted waveform applied to the ultrasound array 2402 does not accidentally depolarize the array. The processor system 2404 generates the depolarization prevention instructions suitable for the ultrasound array 2402 based on the array data that describes the ultrasound array 2402, and the level-shifted waveform from the level shifter 2408 that is currently or previously used for the ultrasound array 2402. In an example, the depolarization prevention instructions include a percentage increase in a level-shifting voltage determined by the processor system 2404 based on the age of the array. For instance, for each year of the array age after five years, the depolarization prevention instructions can instruct the level shifter 2408 to increase the level-shifting voltage by 5%. In another example, the depolarization prevention instructions can instruct the level shifter 2408 to increase the level-shifting voltage by a percentage of the level-shifting voltage that is currently being used by the level shifter 2408.

The calibration system 2400 can operate on an array basis, or an array element basis. Hence, for a PMUT array, the calibration system 2400 can generate the level-shifted waveform based on a first level-shifting voltage, and for a PZT array, the calibration system 2400 can generate the level-shifted waveform based on a second level-shifting voltage. The second level-shifting voltage can be greater than the first level-shifting voltage. In an example, the second level-shifting voltage is at least ten times the first level-shifting voltage. As the ultrasound array 2402 ages, the level-shifting voltage used to generate the level-shifted waveform can be increased by the calibration system 2400 to maintain the performance of the array over its lifespan.

In some embodiments, the waveform generator 2406 generates a driving waveform having a lower voltage swing for some array elements than other array elements. This scheme can be used to effectively change the shape of an array configuration, such as to make the matrix array configuration 2302 approximate the shape of the first circular array configuration 2304, as previously described with respect to FIG. 23. Hence, the calibration system 2400 can be used to enhance the harmonic content of an array to improve a harmonic imaging mode, compared to a conventional system that does not effectively change the shape of an array configuration based on adjustment of a driving waveform.

The calibration system 2400 can be implemented on any suitable device. In some embodiments, the calibration system 2400 is implemented on an ultrasound scanner 2412, which is an example of the scanner 104. Additionally or alternatively, the calibration system 2400 can be implemented on an ultrasound machine 2414, which is an example of the ultrasound machine 102. Hence, in some embodiments, the calibration system 2400 can be enabled before, during, or after an ultrasound examination, e.g., each time the ultrasound system is used for an ultrasound examination. Further, the calibration system 2400 can be enabled periodically, or regularly, at the care facility that operates the ultrasound system. For instance, the calibration system 2400 can be enabled monthly, semi-annually, etc. to ensure that the ultrasound array 2402 does not get accidentally depolarized.

In another example, the calibration system 2400 is implemented on test equipment at a manufacturer 2416 of the ultrasound array 2402. For instance, the calibration system 2400 can be enabled as part of a warranty service, or as part of a subscription-based service paid by the care facility after the warranty of the ultrasound array 2402 expires.

### Example Flow Diagrams

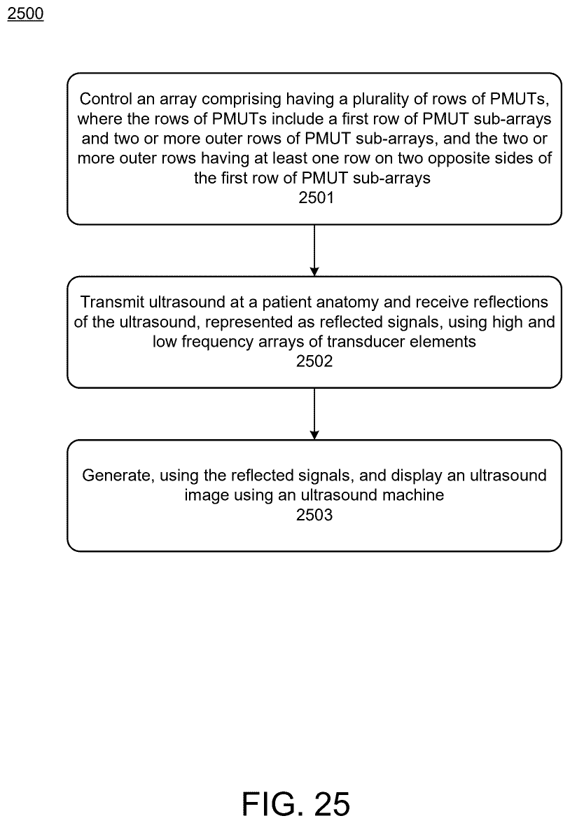

FIG. 25 illustrates some embodiments of an example method 2500 for controlling an ultrasound system. Operations of the method can be performed by processing logic that can comprise hardware (circuitry, dedicated logic, etc.), software (such as is run on a general-purpose computer system or a dedicated machine), firmware, or combinations thereof. The processing logic can be included in an ultrasound system. In some embodiments, the ultrasound system can include an ultrasound scanner, a mobile computing device (e.g., a handset), a display device (e.g., an ultrasound machine), a docking station, and a processor system. In some embodiments, the ultrasound system can include an ultrasound probe, a display device, and a processor system.

Referring to FIG. 25, method 2500 includes controlling an array having a plurality of rows of piezoelectric micromachined ultrasonic transducers (PMUTs), where the rows of PMUTs include a first row of PMUT sub-arrays and two or more outer rows of PMUT sub-arrays, and the two or more outer rows having at least one row on two opposite sides of the first row of PMUT sub-arrays (block 2501). In some embodiments, the PMUT sub-arrays in a first and second rows of PMUT sub-arrays of the one or more outer rows have heights and widths that are different from each other, where the height of each PMUT sub-array corresponding to a lateral dimension that extends in a direction across the array along the two opposite sides and the width corresponding to an elevation dimension perpendicular to the lateral dimension. In some embodiments, at least one of the first and second rows of PMUT sub-arrays comprise PMUT sub-arrays of different heights. In some other embodiments, at least one of the first and second rows of PMUT sub-arrays have the same height as PMUT sub-arrays of the first row of PMUT sub-arrays.

In some embodiments, the first row of PMUT sub-arrays operates at a first ultrasound frequency and the two or more other rows of PMUT sub-arrays operate at a second ultrasound frequency that is different than the first ultrasound frequency. In some embodiments, controlling the array includes controlling a first row of PMUT sub-arrays and two or more outer rows of PMUT sub-arrays independently in one of the modes to operate at the same time by selecting bandwidths and center frequencies of the first row and the two or more rows of PMUT sub-arrays to determine an amount of overlap of the bandwidths of the plurality of PMUT sub-arrays. Such control can include controlling the first row of PMUT sub-arrays and two or more outer rows of PMUT sub-arrays independently in one of the modes to operate at the same time to obtain signals for performing third or higher harmonic imaging by setting frequency of the first row of PMUT sub-arrays to a first ultrasound frequency and setting frequency of the two or more rows of PMUT sub-arrays to be at a harmonic of the first ultrasound frequency. In some embodiments, the harmonic of the first ultrasound frequency is a third harmonic of the first ultrasound frequency. In some other embodiments, such control can include controlling the array including controlling the first row of PMUT sub-arrays and two or more outer rows of PMUT sub-arrays independently in one mode of a set of modes to operate at the same time to obtain signals for performing harmonic imaging by controlling the first row of PMUT sub-arrays to perform a receive operation while controlling the two or more rows of PMUT sub-arrays to perform transmit operations with non-overlapping bandwidths associated with the transmit and receive operations. In some embodiments, the harmonic imaging is third or higher harmonic imaging. In yet some other embodiments, the harmonic imaging is sub-harmonic imaging.

In yet some other embodiments, such control includes controlling the first row of PMUT sub-arrays and two or more outer rows of PMUT sub-arrays independently in one of the modes to operate at the same time to obtain signals for performing harmonic imaging. In some embodiments, this control is performed by controlling the first row of PMUT sub-arrays to perform a receive operation while controlling one row of the two or more rows of PMUT sub-arrays to perform transmit operations with partially overlapping bandwidth responses of the first and one rows of PMUT sub-arrays with an upper limit of a bandwidth of one row of PMUT sub-arrays matching a lower limit of a bandwidth of the first row of PMUT sub-arrays.

In some embodiments, controlling the array includes having the first row of PMUT sub-arrays and two or more outer rows of PMUT sub-arrays operate at the same time or at different times. In some embodiments, the PMUT sub-arrays are part of a scanner or probe having a controller that controls the sub-arrays to operate at the same time or at different times.

Using the PMUT sub-arrays, ultrasound is transmitted at a patient anatomy and receive reflections of the ultrasound by the high and low frequency arrays which are represented as reflected signals based on the mode (block 2502). In some embodiments, depending on the mode, one or both sets of PMUTs (e.g., center and outer rows of PMUTs) transmit ultrasound and one or both arrays receive reflected signals.

Using the reflected signals, an ultrasound image is generated and displayed using an ultrasound machine (block 2503). In some embodiments, a computing device (e.g., an imaging subsystem) of the ultrasound system generates the image based on the received signals.

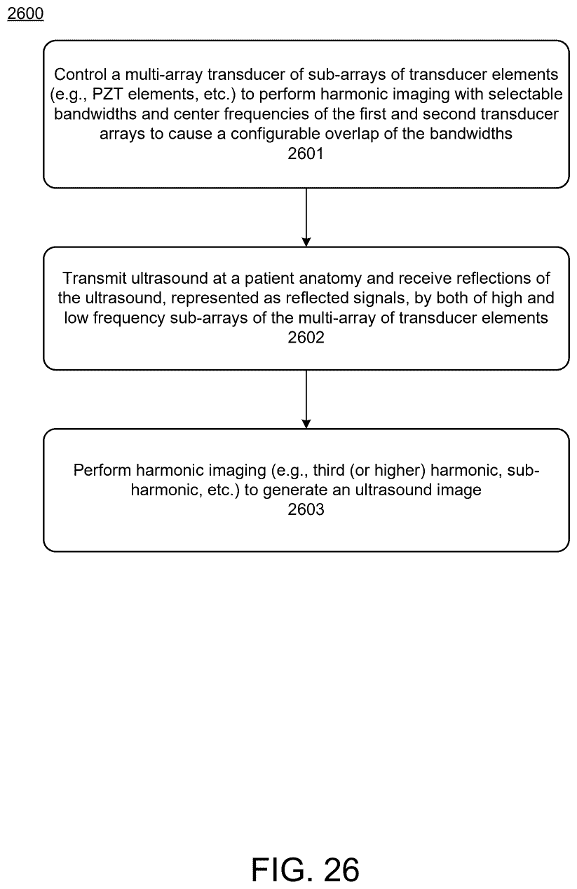

FIG. 26 illustrates some embodiments of an example method 2600 for controlling an ultrasound system. Operations of the method can be performed by processing logic that can comprise hardware (circuitry, dedicated logic, etc.), software (such as is run on a general-purpose computer system or a dedicated machine), firmware, or combinations thereof. The processing logic can be included in an ultrasound system. In some embodiments, the ultrasound system can include an ultrasound scanner, a mobile computing device (e.g., a handset), a display device (e.g., an ultrasound machine), a docking station, and a processor system. In some embodiments, the ultrasound system can include an ultrasound probe, a display device, and a processor system.

Referring to FIG. 26, method 2600 includes controlling a multi-array transducer of sub-arrays of transducer elements (e.g., PZT elements, etc.) to perform harmonic imaging with selectable bandwidths and center frequencies of the first and second transducer arrays to cause a configurable overlap of the bandwidths (block 2601). In some embodiments, at least first and transducer arrays of the transducer sub-arrays have heights and widths that are different from each other, where the height of each transducer sub-array corresponding to a lateral dimension across the array and the width transducer sub-array corresponding to an elevation dimension perpendicular to the lateral dimension. In some embodiments, controlling the multi-array transducer includes operating a first transducer sub-array at a first ultrasound frequency and to transmit ultrasound and to operate the second transducer sub-array at a second ultrasound frequency that is different from the first ultrasound frequency to receive reflections of the transmitted ultrasound. In some embodiments, the first and second transducer sub-arrays can include PMUT array elements, PZT array elements, and/or CMUT array elements. In some embodiments, the multi-array transducer is part of a scanner or probe having a controller that controls the array to operate in this way.

In some embodiments, the multi-array transducer includes a matrix array configuration in which the first transducer sub-array is surrounded by and centrally-located with respect to one or more other transducer sub-arrays in the multi-array transducer including the second transducer sub-array. In some embodiments of the matrix array configuration, the array elements of the first transducer sub-array are smaller than array elements of the second transducer sub-array.

In some other embodiments, the multi-array transducer includes a circular array configuration with an inner transducer sub-array and an outer transducer sub-array. The inner transducer sub-array can have rings of circular array elements of different sizes, while the outer transducer sub-array has circular array elements. In some embodiments, each of the array elements are centered at the same distance from a center of the inner transducer sub-array.

In yet some other embodiments, the multi-array transducer includes a circular array configuration having an inner transducer sub-array and two outer transducer sub-arrays. The inner transducer sub-array can include elliptically-shaped array elements of different sizes, while the outer transducer sub-arrays are centered at the same distance from the center of the inner transducer array and have circular array elements of different size from one another. These circular array elements are spaced at the distance so that the larger array elements of the outer transducer array do not touch the smaller array elements of the outer transducer array.

In still some other embodiments, the multi-array transducer includes an octagon array configuration having a first transducer array with octagonal array elements and a second transducer array with square array elements. The square array elements of the second transducer array can be located such that each side of a square array element is adjacent to a side of octagonal array elements of the first transducer array.

In some further embodiments, the multi-array transducer includes a hexagon array configuration having a first transducer array of hexagonally-shaped elements and a second transducer array of hexagonally-shaped elements. The array elements of the first and second transducer arrays can be arranged in a hexagonal closest packing configuration such that array elements of the first transducer sub-array abut one another and the array elements of the second transducer sub-array do not abut one another and abut array elements of the first transducer sub-array. In some embodiments, the first transducer sub-array includes array elements of a first type, operates at the first ultrasound frequency, and has a first bandwidth, while the second transducer sub-array has array elements of a second type, operate at the second ultrasound frequency, and has a second bandwidth.

In some additional embodiments, the multi-array transducer includes an Einstein tile array configuration having a first sub-array and a second sub-array, where each of the first and second arrays have array elements shaped as Einstein tiles.

Using the multi-array transducer of transducer elements, ultrasound is transmitted at a patient anatomy and receive reflections of the ultrasound by both of the high and low frequency sub-arrays which are represented as reflected signals (block 2602). In some embodiments, both sub-arrays (e.g., center and outer rows of PZT, PMUTs and/or CMUTs) receive reflected signals.

Based on the received signals, harmonic imaging is performed (block 2603). In some embodiments, a computing device (e.g., an imaging subsystem) of the ultrasound system performs sub-harmonic imaging based on the received signals. In some other embodiments, a computing device (e.g., an imaging subsystem) of the ultrasound system performs third (or higher) harmonic imaging based on the received signals. The result of performing the harmonic imaging is the display of an image generated using an ultrasound machine.

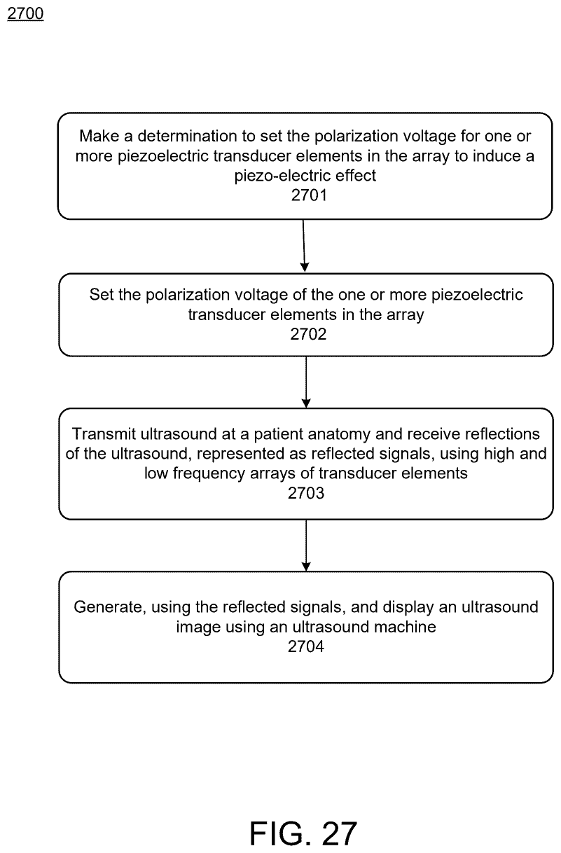

FIG. 27 illustrates some embodiments of an example method 2700 for controlling an ultrasound system. Operations of the method can be performed by processing logic that can comprise hardware (circuitry, dedicated logic, etc.), software (such as is run on a general-purpose computer system or a dedicated machine), firmware, or combinations thereof. The processing logic can be included in an ultrasound system. In some embodiments, the ultrasound system can include an ultrasound scanner, a mobile computing device (e.g., a handset), a display device (e.g., an ultrasound machine), a docking station, and a processor system. In some embodiments, the ultrasound system can include an ultrasound probe, a display device, and a processor system.

Referring to FIG. 27, a determination is made to set the polarization voltage for one or more piezoelectric transducer elements in the array to induce a piezo-electric effect (block 2701). In some embodiments, the determination is made after a determination that the one or more piezoelectric transducer elements have become depolarized. In some other embodiments, the determination is made based on time (e.g., automatically at periodic time intervals (e.g., monthly, yearly, etc.), an amount of time that has elapsed since the last application of the polarization voltage, etc.).

Method 2700 includes setting the polarization voltage of the one or more piezoelectric transducer elements in the array (block 2702). In some embodiments, setting the polarization voltage includes applying a voltage to the one or more piezoelectric transducer elements in the array. The application of the voltage can include increasing the polarization voltage (e.g., over time, etc.). The setting of the polarization voltage can include shifting a driving waveform on a per element basis based on element type, such as described above. The setting of the polarization voltage can be performed by a voltage control circuit, which can be part of level-shifting depolarization circuitry.

Using the array with one or more transducer elements re-polarized, ultrasound is transmitted at a patient anatomy and receive reflections of the ultrasound by the high and low frequency arrays which are represented as reflected signals based on the mode (block 2703). In some embodiments, depending on the mode, one or more sub-arrays of transducer elements (e.g., center and outer rows (sub-arrays) of PZTs and/or PMUTs) transmit ultrasound and one or both arrays receive reflected signals.

Using the reflected signals, an ultrasound image is generated and displayed using an ultrasound machine (block 2704). In some embodiments, a computing device (e.g., an imaging subsystem) of the ultrasound system generates the image based on the received signals.

There are a number of example embodiments described herein.

Example 1 is an ultrasound device including: a lens; and array coupled to the lens; and a controller. The array has a plurality of rows of ultrasonic transducer elements, with the plurality of rows of transducer elements having a first row of transducer element sub-arrays and two or more outer rows of transducer element sub-arrays. The two or more outer rows have at least one row on two opposite sides of the first row of transducer element sub-arrays, and transducer element sub-arrays in first and second rows of transducer element sub-arrays of the one or more outer rows have heights and widths that are different from each other, with the height of each transducer element sub-array corresponding to a lateral dimension and the width corresponding to an elevation dimension perpendicular to the lateral dimension. The controller is coupled to the array and configured to control the first row of transducer element sub-arrays and two or more outer rows of transducer element sub-arrays to operate at a same time or at different times.

Example 2 is the ultrasound device of example 1 that may optionally include that the transducer elements comprise piezoelectric micromachined ultrasonic transducers (PMUTs), and the plurality of rows of transducer elements comprise a plurality of rows of PMUTs having a first row of PMUT sub-arrays and two or more outer rows of PMUT sub-arrays.

Example 3 is the ultrasound device of example 1 that may optionally include that at least one of the first and second rows of transducer element sub-arrays comprise transducer element sub-arrays of different heights.

Example 4 is the ultrasound device of example 1 that may optionally include that at least one of the first and second rows of transducer element sub-arrays have a same height as transducer element sub-arrays of the first row of sub-arrays.

Example 5 is the ultrasound device of example 1 that may optionally include that the first row of transducer element sub-arrays operates at a first ultrasound frequency and the two or more other rows of transducer element sub-arrays operate at a second ultrasound frequency that is different than the first ultrasound frequency.

Example 6 is the ultrasound device of example 5 that may optionally include that the controller is configured to control the first row of transducer element sub-arrays and two or more outer rows of transducer element sub-arrays independently in one of the modes to operate at the same time by selecting bandwidths and center frequencies of the first row and the two or more rows of transducer element sub-arrays to determine an amount of overlap of the bandwidths of the plurality of transducer element sub-arrays.

Example 7 is the ultrasound device of example 6 that may optionally include that the controller is configured to control the first row of transducer element sub-arrays and two or more outer rows of transducer element sub-arrays independently in one of the modes to operate at the same time to obtain signals for performing third or higher harmonic imaging by setting frequency of the first row of transducer element sub-arrays to a first ultrasound frequency and setting frequency of the two or more rows of transducer element sub-arrays to be at a harmonic of the first ultrasound frequency.

Example 8 is the ultrasound device of example 7 that may optionally include that the harmonic of the first ultrasound frequency is a third harmonic of the first ultrasound frequency.

Example 9 is the ultrasound device of example 6 that may optionally include that the controller is configured to control the first row of transducer element sub-arrays and two or more outer rows of transducer element sub-arrays independently in one of the modes to operate at the same time to obtain signals for performing harmonic imaging by controlling the first row of transducer element sub-arrays to perform a receive operation while controlling the two or more rows of transducer element sub-arrays to perform transmit operations with non-overlapping bandwidths associated with the transmit and receive operations.

Example 10 is the ultrasound device of example 9 that may optionally include that the harmonic imaging is third or higher harmonic imaging.

Example 11 is the ultrasound device of example 9 that may optionally include that the harmonic imaging is sub-harmonic imaging.

Example 12 is the ultrasound device of example 6 that may optionally include that the controller is configured to control the first row of transducer element sub-arrays and two or more outer rows of transducer element sub-arrays independently in one of the modes to operate at the same time to obtain signals for performing harmonic imaging by controlling the first row of transducer element sub-arrays to perform a receive operation while controlling one row of the two or more rows of transducer element sub-arrays to perform transmit operations with partially overlapping bandwidth responses of the first and one rows of transducer element sub-arrays with an upper limit of a bandwidth of one row of transducer element sub-arrays matching a lower limit of a bandwidth of the first row of transducer element sub-arrays.

Example 13 is the ultrasound device of example 1 that may optionally include a voltage control circuit to set a polarization voltage for one or more elements of the plurality of transducer element sub-arrays.

Example 14 is the ultrasound device of example 13 that may optionally include that the voltage control circuit is part of level-shifting depolarization circuitry responsive to depolarization of one or more elements of the plurality of transducer element sub-arrays.

Example 15 is the ultrasound device of example 13 that may optionally include that the voltage control circuit operates automatically at periodic time intervals.

Example 16 is the ultrasound device of example 1 that may optionally include that the transducer elements comprise capacitive micromachined ultrasonic transducer (CMUT), and the plurality of rows of transducer elements comprise a plurality of rows of CMUTs having a first row of CMUT sub-arrays and two or more outer rows of CMUT sub-arrays.

Example 17 is an ultrasound device including a lens and a multi-array transducer coupled to the lens and having a plurality of transducer sub-arrays, where at least first and transducer arrays of the plurality of transducer arrays have heights and widths that are different from each other. The height of each transducer array corresponds to a lateral dimension across the array and the width transducer array corresponding to an elevation dimension perpendicular to the lateral dimension. The ultrasound device also includes a controller coupled to the array and configured to control the plurality of transducer sub-arrays to operate at a same time or at different times and to perform harmonic imaging with selectable bandwidths and center frequencies of first and second transducer sub-arrays to cause a configurable overlap of the bandwidths, the first transducer sub-array controlled to operate at a first ultrasound frequency and to transmit ultrasound and the second transducer sub-array controlled to operate at a second ultrasound frequency, different from the first ultrasound frequency and to receive reflections of the ultrasound.

Example 18 is the ultrasound device of example 17 that may optionally include that the first transducer sub-array includes at least one of piezoelectric micromachined ultrasonic transducer (PMUT) array elements and lead zirconate titanate (PZT) array elements, and the second transducer sub-array includes capacitive micromachined ultrasonic transducer (CMUT) array elements.

Example 19 is the ultrasound device of example 17 that may optionally include that the multi-array transducer comprises a matrix array configuration in which the first transducer sub-array is surrounded by and centrally-located with respect to one or more other transducer sub-array of the plurality of transducer sub-arrays including the second transducer sub-array, and array elements of the first transducer sub-array are smaller than array elements of the second transducer sub-array.

Example 20 is the ultrasound device of example 17 that may optionally include that the multi-array transducer comprises a circular array configuration with an inner transducer sub-array and an outer transducer sub-array, the inner transducer sub-array having rings of circular array elements of different sizes, and the outer transducer sub-array having circular array elements each of a same size that are centered at a same distance from a center of the inner transducer sub-array.

Example 21 is the ultrasound device of example 17 that may optionally include that the multi-array transducer comprises a circular array configuration having an inner transducer sub-array and two outer transducer sub-arrays, the inner transducer sub-array including elliptically-shaped array elements of different sizes, the outer transducer sub-arrays being centered at a same distance from a center of the inner transducer sub-array and having different size circular array elements from one another that are spaced at the distance so that larger array elements of the outer transducer sub-array do not touch smaller array elements of the outer transducer sub-array.

Example 22 is the ultrasound device of example 17 that may optionally include that the multi-array transducer includes an octagon array configuration having a first transducer sub-array with octagonal array elements and a second transducer array with square array elements, the square array elements of the second transducer sub-array being located such that each side of a square array element is adjacent to a side of octagonal array elements of the first transducer sub-array.

Example 23 is the ultrasound device of example 17 that may optionally include that the multi-array transducer includes a hexagon array configuration having a first transducer sub-array of hexagonally-shaped elements and a second transducer sub-array of hexagonally-shaped elements, the array elements of the first and second transducer sub-arrays being arranged in a hexagonal closest packing configuration such that array elements of the first transducer sub-array abut one another. The array elements of the second transducer sub-array do not abut one another and abut array elements of the first transducer sub-array. The first transducer sub-array includes array elements of a first type, operates at the first ultrasound frequency, and has a first bandwidth, and the second transducer sub-array has array elements of a second type, operate at the second ultrasound frequency, and has a second bandwidth.

Example 24 is the ultrasound device of example 17 that may optionally include that the multi-array transducer includes an Einstein tile array configuration having a first sub-array and a second sub-array, each having array elements shaped as Einstein tiles.

Example 25 is the ultrasound device of example 17 that may optionally include that the harmonic imaging is third harmonic or sub-harmonic imaging.

Example 26 is an ultrasound device including: an array of transducer elements; and level-shifting depolarization circuitry coupled to the array to set a polarization voltage for one or more piezoelectric transducer elements in the array to induce a piezo-electric effect.

Example 27 is the ultrasound device of example 26 that may optionally include that the transducer elements are piezoelectric transducer (PZT) elements.

Example 28 is the ultrasound device of example 26 that may optionally include that the transducer elements are PMUT elements.

Example 29 is the ultrasound device of example 26 that may optionally include that the polarization voltage is proportional to thickness of material of the array.

Example 30 is the ultrasound device of example 26 that may optionally include that the level-shifting depolarization circuitry operates automatically at periodic time intervals.

Example 31 is the ultrasound device of example 26 that may optionally include that level-shifting depolarization circuitry includes a voltage control circuit to increase the polarization voltage over time.

Example 32 is the ultrasound device of example 26 that may optionally include that the level-shifting depolarization circuitry level shifts a driving waveform on a per element basis based on element type.

Example 33 is the ultrasound device of example 26 that may optionally include that the piezoelectric transducer (PZT) elements are part of one or more PMUTs.

Example 34 is a method that performs one of more of the operations described above in Examples 1-33.

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in some embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware (e.g., ASICs or FPGA devices), computer software that runs on computer hardware, or combinations of both. Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the rendering techniques described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without other input or prompting, whether these features, elements or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

We claim:

1. An ultrasound device comprising:
a lens;
an array coupled to the lens and having a plurality of rows of ultrasonic transducer elements, the plurality of rows of transducer elements having a first row of transducer element sub-arrays and two or more outer rows of transducer element sub-arrays, the two or more outer rows having at least one row on two opposite sides of the first row of transducer element sub-arrays, wherein transducer element sub-arrays in first and second rows of transducer element sub-arrays of the one or more outer rows have heights and widths that are different from each other, the height of each transducer element sub-array corresponding to a lateral dimension and the width corresponding to an elevation dimension perpendicular to the lateral dimension; and
a controller coupled to the array and configured to control the first row of transducer element sub-arrays and two or more outer rows of transducer element sub-arrays to operate at a same time or at different times.

2. The ultrasound device of claim 1 wherein the transducer elements comprise piezoelectric micromachined ultrasonic transducers (PMUTs), and the plurality of rows of transducer elements comprise a plurality of rows of PMUTs having a first row of PMUT sub-arrays and two or more outer rows of PMUT sub-arrays.

3. The ultrasound device of claim 1 wherein at least one of the first and second rows of transducer element sub-arrays comprise transducer element sub-arrays of different heights.

4. The ultrasound device of claim 1 wherein at least one of the first and second rows of transducer element sub-arrays have a same height as transducer element sub-arrays of the first row of sub-arrays.

5. The ultrasound device of claim 1 wherein the first row of transducer element sub-arrays operates at a first ultrasound frequency and the two or more other rows of transducer element sub-arrays operate at a second ultrasound frequency that is different than the first ultrasound frequency.

6. The ultrasound device of claim 5 wherein the controller is configured to control the first row of transducer element sub-arrays and two or more outer rows of transducer element sub-arrays independently in one of the modes to operate at the same time by selecting bandwidths and center frequencies of the first row and the two or more rows of transducer element sub-arrays to determine an amount of overlap of the bandwidths of the plurality of transducer element sub-arrays.

7. The ultrasound device of claim 6 wherein the controller is configured to control the first row of transducer element sub-arrays and two or more outer rows of transducer element sub-arrays independently in one of the modes to operate at the same time to obtain signals for performing third or higher harmonic imaging by setting frequency of the first row of transducer element sub-arrays to a first ultrasound frequency and setting frequency of the two or more rows of transducer element sub-arrays to be at a harmonic of the first ultrasound frequency.

8. The ultrasound device of claim 7 wherein the harmonic of the first ultrasound frequency is a third harmonic of the first ultrasound frequency.

9. The ultrasound device of claim 6 wherein the controller is configured to control the first row of transducer element sub-arrays and two or more outer rows of transducer element sub-arrays independently in one of the modes to operate at the same time to obtain signals for performing harmonic imaging by controlling the first row of transducer element sub-arrays to perform a receive operation while controlling the two or more rows of transducer element sub-arrays to perform transmit operations with non-overlapping bandwidths associated with the transmit and receive operations.

10. The ultrasound device of claim 9 wherein the harmonic imaging is third or higher harmonic imaging.

11. The ultrasound device of claim 9 wherein the harmonic imaging is sub-harmonic imaging.

12. The ultrasound device of claim 6 wherein the controller is configured to control the first row of transducer element sub-arrays and two or more outer rows of transducer element sub-arrays independently in one of the modes to operate at the same time to obtain signals for performing harmonic imaging by controlling the first row of transducer element sub-arrays to perform a receive operation while controlling one row of the two or more rows of transducer element sub-arrays to perform transmit operations with partially overlapping bandwidth responses of the first and one rows of transducer element sub-arrays with an upper limit of a bandwidth of one row of transducer element sub-arrays matching a lower limit of a bandwidth of the first row of transducer element sub-arrays.

13. The ultrasound device of claim 1 further comprising a voltage control circuit to set a polarization voltage for one or more elements of the plurality of transducer element sub-arrays.

14. The ultrasound device of claim 13 wherein the voltage control circuit is part of level-shifting depolarization circuitry responsive to depolarization of one or more elements of the plurality of transducer element sub-arrays.

15. The ultrasound device of claim 13 wherein the voltage control circuit operates automatically at periodic time intervals.

16. The ultrasound device of claim 1 wherein the transducer elements comprise capacitive micromachined ultrasonic transducer (CMUT), and the plurality of rows of transducer elements comprise a plurality of rows of CMUTs having a first row of CMUT sub-arrays and two or more outer rows of CMUT sub-arrays.

17. An ultrasound device comprising:
a lens;
a multi-array transducer coupled to the lens and having a plurality of transducer sub-arrays, wherein at least first and transducer arrays of the plurality of transducer arrays have heights and widths that are different from each other, the height of each transducer array corresponding to a lateral dimension across the array and the width transducer array corresponding to an elevation dimension perpendicular to the lateral dimension; and
a controller coupled to the array and configured to control the plurality of transducer sub-arrays to operate at a same time or at different times and to perform harmonic imaging with selectable bandwidths and center frequencies of first and second transducer sub-arrays to cause a configurable overlap of the bandwidths, the first transducer sub-array controlled to operate at a first ultrasound frequency and to transmit ultrasound and the second transducer sub-array controlled to operate at a second ultrasound frequency, different from the first ultrasound frequency and to receive reflections of the ultrasound.

18. The ultrasound device as described in claim 17, wherein the multi-array transducer comprises:
a matrix array configuration in which the first transducer sub-array is surrounded by and centrally-located with respect to one or more other transducer sub-array of the plurality of transducer sub-arrays including the second transducer sub-array, and array elements of the first transducer sub-array are smaller than array elements of the second transducer sub-array;
a circular array configuration with an inner transducer sub-array and an outer transducer sub-array, the inner transducer sub-array having rings of circular array elements of different sizes, and the outer transducer sub-array having circular array elements each of a same size that are centered at a same distance from a center of the inner transducer sub-array; or
a circular array configuration having an inner transducer sub-array and two outer transducer sub-arrays, the inner transducer sub-array including elliptically-shaped array elements of different sizes, the outer transducer sub-arrays being centered at a same distance from a center of the inner transducer sub-array and having different size circular array elements from one another that are spaced at the distance so that larger array elements of the outer transducer sub-array do not touch smaller array elements of the outer transducer sub-array.

19. The ultrasound device as described in claim 17, wherein the multi-array transducer comprises:
an octagon array configuration having a first transducer sub-array with octagonal array elements and a second transducer array with square array elements, the square array elements of the second transducer sub-array being located such that each side of a square array element is adjacent to a side of octagonal array elements of the first transducer sub-array;
a hexagon array configuration having a first transducer sub-array of hexagonally-shaped elements and a second transducer sub-array of hexagonally-shaped elements, the array elements of the first and second transducer sub-arrays being arranged in a hexagonal closest packing configuration such that array elements of the first transducer sub-array abut one another, and the array elements of the second transducer sub-array do not abut one another and abut array elements of the first transducer sub-array, wherein the first transducer sub-array includes array elements of a first type, operates at the first ultrasound frequency, and has a first bandwidth, and the second transducer sub-array has array elements of a second type, operate at the second ultrasound frequency, and has a second bandwidth; or
an Einstein tile array configuration having a first sub-array and a second sub-array, each having array elements shaped as Einstein tiles.

20. The ultrasound device as described in claim 17, wherein the harmonic imaging is third harmonic or sub-harmonic imaging.
